(19) United States
(12) Patent Application Publication
(10) Pub. No.: US 2010/0184804 A1
(43) Pub. Date: Jul. 22, 2010

(54) METHODS FOR THE PREVENTION OR TREATMENT OF BACTERIAL AND FUNGAL INFECTIONS

(76) Inventors: Laurence G. Rahme, Brookline, MA (US); Hui Cao, Natick, MA (US); Francois Lepine, Montreal (CA); Eric Deziel, Somerville, MA (US); Ronald G. Tompkins, Boston, MA (US)

Correspondence Address: CLARK & ELBING LLP, 101 FEDERAL STREET, BOSTON, MA, 02110, US

(21) Appl. No.: 12/623,307

(22) Filed: Nov. 20, 2009

Related U.S. Application Data
(63) Continuation of application No. 10/493,989, filed on Nov. 16, 2004, now abandoned.
(60) Provisional application No. 60/385,026, filed on May 31, 2002.
(60) Provisional application No. 60/340,063, filed on Oct. 30, 2001.

(51) Int. Cl. A61K 31/47; A61K 31/335; C12Q 1/02; A61P 31/04

(52) U.S. Cl. 514/312; 514/449; 435/29

(57) ABSTRACT

The present invention features improved methods for treating, stabilizing, or preventing a bacterial or a fungal infection in a plant or an animal, such as a mammal. In particular, these methods involve the use of a compound, that is controlled by MvfR, and that affects the expression of an MvfR protein or that promotes its modification or inactivation, or a compound produced by P. aeruginosa strain PA14, but not by P. aeruginosa containing an mvfR mutation, in late stationary phase cultures.

### FIELD OF THE INVENTION

The field of the invention is prevention and treatment of bacterial and fungal infections.

### BACKGROUND OF THE INVENTION

Pseudomonas aeruginosa (P. aeruginosa) is an opportunistic pathogen that can infect both animals and plants. The pathophysiology of infections due to P. aeruginosa is complex, as shown by the clinical diversity of the diseases associated with this organism and by the multiplicity of cell-associated and secreted virulence factors it produces (Lyczak et al., Microbes and Infection 2:1051-1060, 2000). In humans, P. aeruginosa is responsible for persistent infections in immuno-compromised patients, including cancer patients subjected to chemo- or radiation-therapies, burn patients, patients with AIDS, and patients undergoing bone marrow transplantation (Fink, “Pseudomonas aeruginosa the Opportunist: Pathogenesis and Disease,” pp. 1-5, ed. Fink, R. B., Jr. (CRC Press, Boca Raton), 1993). P. aeruginosa also is found in the lungs of over 80% of cystic fibrosis patients over 26 years of age (Fitzsimmons, J. Pediatr. 122:1-9, 1993) and is the leading cause of nosocomial infections and hospital-acquired pneumonia (Jarvis and Martone, J. Antimicrob. Chemother. 29:S19-S24, 1992). In addition to being involved in a number of human diseases, several characteristics of P. aeruginosa make it difficult to control effectively, including its highly impermeable membrane (Hancock, Clin. Infect. Dis. 1:S93-S99, 1998), the presence of β-lactamase (Hancock and Woodruff, Rev. Infect. Dis. 10:770-775, 1988; Philippon et al., Antimicrob. Agents Chemother. 41:2188-2195, 1997), and its various efflux systems (Kohler et al., Mol. Microbiol. 23:345-354, 1997; Poole et al., J. Bacteriol. 175:7363-7372, 1993; Poole et al., Mol. Microbiol. 21:713-724, 1996).

Despite a detailed knowledge of some of the extracellular proteins and several surface-associated components identified in P. aeruginosa, the understanding of the pathogenic nature of P. aeruginosa infections is rudimentary.

### SUMMARY OF THE INVENTION

The present invention features improved methods for treating, stabilizing, or preventing a bacterial or fungal infection in a plant or an animal, such as a mammal. In particular, these methods involve the use of compounds that affect the expression of an MvfR protein or promote its modification, e.g., cleavage, post-translational modification, or inactivation, or compounds produced by P. aeruginosa strain PA14 in late stationary phase cultures, but not by P. aeruginosa containing an mvfR mutation. These compounds may also inhibit or decrease the virulence of P. aeruginosa strain PA14.

Accordingly, the first aspect of the invention features a method of treating, stabilizing, or preventing a bacterial or a fungal infection in a mammal. This method includes administering, to the mammal, a compound that promotes the modification, e.g., the cleavage or post-translational modification, of an MvfR protein in an amount sufficient to treat, stabilize, or prevent the bacterial infection. In addition, a second aspect of the invention features a method of treating, stabilizing, or preventing a bacterial infection in a plant. This method includes administering, to the plant, a compound that promotes the modification, e.g., the cleavage or post-translational modification, of an MvfR protein in an amount sufficient to treat, stabilize, or prevent the bacterial infection. In preferred embodiments of these aspects, the MvfR protein is cleaved between amino acids 146 and 147 and/or the cleavage of an MvfR protein results in a polypeptide fragment with a molecular weight of approximately 22 kDa.

The third aspect of the invention features another method of treating, stabilizing, or preventing a bacterial or a fungal infection in a mammal. This method involves administering a compound, to the mammal, in an amount sufficient to treat, stabilize, or prevent the bacterial infection, where this compound is (a) produced by Pseudomonas aeruginosa strain PA14; (b) produced at greater levels by a Pseudomonas aeruginosa strain PA14 having a wild-type mvfR nucleic acid than by a Pseudomonas aeruginosa strain PA14 having an mvfR mutation, under the identical growth conditions, where the mvfR mutation results in the inactivation of an MvfR protein encoded by a nucleic acid sequence including an mvfR mutation, for example, an mvfR mutation that results in a substitution of a stop codon for the mvfR codon encoding MvfR amino acid Glu 151; and (c) produced at greater levels by Pseudomonas aeruginosa strain PA14 during late stationary phase than during exponential phase.

In a fourth aspect, the invention features another method of treating, stabilizing, or preventing a bacterial or a fungal infection in a plant. This method involves administering a compound, to the plant, in an amount sufficient to treat, stabilize, or prevent the bacterial infection, where this compound is (a) produced by Pseudomonas aeruginosa strain PA14; (b) produced at greater levels by a Pseudomonas aeruginosa strain PA14 having a wild-type mvfR nucleic acid than by a Pseudomonas aeruginosa strain PA14 having an mvfR mutation, under the identical growth conditions, where the mvfR mutation results in the inactivation of an MvfR protein encoded by a nucleic acid sequence including an mvfR mutation, for example, an mvfR mutation that results in a substitution of a stop codon for the mvfR codon encoding MvfR amino acid Glu 151; and (c) produced at greater levels by Pseudomonas aeruginosa strain PA14 during late stationary phase than during exponential phase.

In a preferred embodiment of the first four aspects of the invention, the compound is more soluble in ethyl acetate than in water.

The fifth aspect of the invention features a further method of treating, stabilizing, or preventing a bacterial or a fungal infection in a mammal. This method encompasses administering a compound to the mammal in an amount sufficient to treat, stabilize, or prevent the bacterial infection, where this compound is capable of being isolated by an ethyl acetate extraction of the supernatant of a Pseudomonas aeruginosa strain PA14 culture in late stationary phase. Furthermore, the sixth aspect of the invention features a further method of treating, stabilizing, or preventing a bacterial infection in a plant. This method encompasses administering a compound to the plant in an amount sufficient to treat, stabilize, or prevent the bacterial infection, where this compound is capable of being isolated by an ethyl acetate extraction of the supernatant of a Pseudomonas aeruginosa strain PA14 culture in late stationary phase.

In preferred embodiments of the first six aspects of the invention, the bacterial infection is an infection by a Gram-negative bacterium, for example a Pseudomonas infection. In a preferred embodiment of the first, third, and fifth aspect of the invention, the bacterial infection may be by a Pseudomonas aeruginosa strain. In addition, the compound may be a homoserine lactone such as N-(3-oxododecanoyl)-L-homoserine lactone or N-butyryl-L-homoserine lactone, or it may be 2-heptyl-3-hydroxy-4-quinolone.

Furthermore, in additional preferred embodiments of the first, third, and fifth aspects of the invention, the mammal, for example, a human, may be immuno-compromised or may have cystic fibrosis.

A seventh aspect of the invention features a method of purifying a compound that induces the modification, e.g., the cleavage or post-translational modification, of an MvfR protein. This method involves preferentially dissolving, in an organic solvent, a compound from the supernatant of a Pseudomonas aeruginosa culture, for example, a culture of Pseudomonas aeruginosa strain PA14, in late stationary phase, where this compound has the ability to induce the modification, e.g., the cleavage or post-translational modification, of an MvfR protein.

In a preferred embodiment of the seventh aspect of the invention, this Pseudomonas aeruginosa has a naturally-occurring mvfR nucleic acid sequence. In another embodiment, the organic solvent is ethyl acetate. Furthermore, the compound may be a homoserine lactone, for example N-(3-oxododecanoyl)-L-homoserine lactone or N-butyryl-L-homoserine lactone, or the compound may be 2-heptyl-3-hydroxy-4-quinolone, or one of the following compounds:

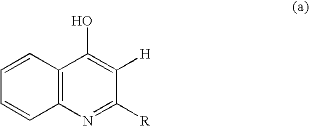

where R=C₅H₁₁, C₇H₁₅, C₉H₁₉, or C₁₁H₂₃ and/or the compound has an M+H ion of 216, 244, 272, or 300 daltons;

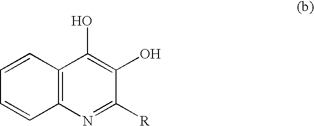

where R=C₅H₁₁, C₇H₁₅, or C₉H₁₉ and/or the compound has an M+H ion of 232, 260, or 288 daltons;

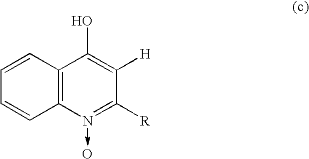

where R=C₅H₁₁, C₇H₁₅, C₉H₁₉, or C₁₁H₂₃ and/or the compound has an M+H ion of 232, 260, 288, or 316 daltons;

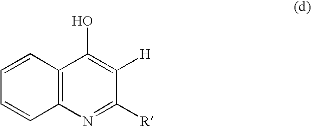

where R′=C₉H₁₇, or C₁₁H₂₁ and/or the compound has an M+H ion of 270 or 298 daltons; or

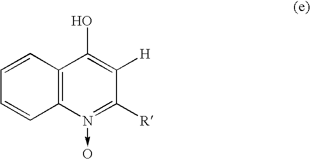

where R′=C₇H₁₃, C₉H₁₇, or C₁₁H₂₁ and/or the compound has an M+H ion of 258, 286, or 314 daltons.

In a further embodiment of this aspect, the compound is produced at greater levels by a wild-type Pseudomonas aeruginosa strain PA14 than by a Pseudomonas aeruginosa strain PA14 comprising an mvfR, under the identical growth conditions, where the mvfR mutation results in the inactivation of an MvfR protein encoded by a nucleic acid sequence including an mvfR mutation, for example, an mvfR mutation that results in a substitution of a stop codon for the mvfR codon encoding MvfR amino acid Glu 151. In addition, this compound may be produced at greater levels by Pseudomonas aeruginosa strain PA14 during late stationary phase than during early stationary phase.

An eighth aspect of the invention is a screening method for determining whether a compound promotes the modification, e.g., the cleavage or post-translational modification, of the MvfR protein. This method includes the steps of (a) contacting a cell expressing an MvfR protein with a candidate compound, and (b) measuring the amount of modified MvfR protein, where an increase in modified MvfR protein indicates that the candidate compound promotes the modification, e.g., the cleavage or post-translational modification, of the MvfR protein.

In a preferred embodiment, step (b) of the eighth aspect of the invention involves measuring the amount of cleaved MvfR protein secreted by the cell, for example, a Pseudomonas aeruginosa cell, such as one from Pseudomonas aeruginosa strain PA14. In other embodiments of this aspect, the compound is a peptide or an autoinducer.

In a ninth aspect, the invention features a method of treating, stabilizing, or preventing a bacterial or fungal infection in a mammal. This method involves administering to the mammal (e.g., a human) one or more compounds listed in Table 4 in an amount sufficient to treat, stabilize, or prevent the infection. In preferred embodiments, the compound promotes the modification, e.g., the cleavage or post-translational modification, of an MvfR protein in an amount sufficient to treat, stabilize, or prevent the infection. It is also contemplated that the compound may function through a different mechanism to inhibit virulence or that another compound present in the supernatant functions to inhibit virulence. In some embodiments, the mammal is immuno-compromised or has cystic fibrosis. In certain embodiments, the infection is a Pseudomonas aeruginosa infection.

In a tenth aspect, the invention features a method of treating, stabilizing, or preventing a bacterial or fungal infection in a plant. This method administering to the plant one or more compounds listed in Table 4 in an amount sufficient to treat, stabilize, or prevent the infection. In preferred embodiments, compound that promotes the modification, e.g., the cleavage or post-translational modification, of an MvfR protein in an amount sufficient to treat, stabilize, or prevent said infection. In certain embodiments, the infection is a Pseudomonas aeruginosa infection.

In an eleventh aspect, the invention features a method of synthesizing or quantifying PQS, a derivative of PQS, or an analog of PQS. This method involves reacting one of the PQS precursors listed in FIG. 21 or one of the following compounds:

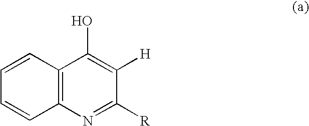

where R=C₅H₁₁, C₇H₁₅, C₉H₁₉, or C₁₁H₂₃ and/or the compound has an M+H ion of 216, 244, 272, or 300 daltons;

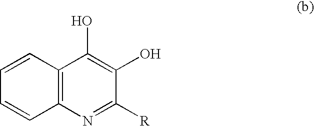

where R=C₅H₁₁, C₇H₁₅, or C₉H₁₉ and/or the compound has an M+H ion of 232, 260, or 288 daltons;

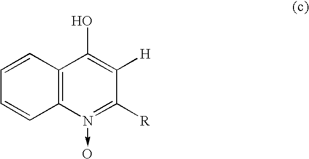

where R=C₅H₁₁, C₇H₁₅, C₉H₁₉, or C₁₁H₂₃ and/or the compound has an M+H ion of 232, 260, 288, or 316 daltons;

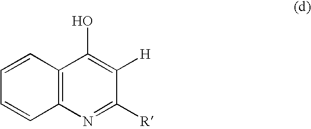

where R′=C₉H₁₇, or C₁₁H₂₁ and/or the compound has an M+H ion of 270 or 298 daltons; or

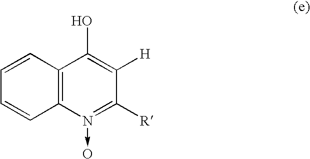

where R′=C₇H₁₃, C₉H₁₇, or C₁₁H₂₁ and/or the compound has an M+H ion of 258, 286, or 314 daltons. For example, the method may involve one or more of the reactions listed in FIG. 21. These compounds may be synthesized in vitro or in vivo.

In preferred embodiments of any of the various aspects of the invention, the mass spectrum of the compound contains a M+H ion of 216, 232, 244, 258, 260, 270, 272, 286, 288, 298, 300, 314, or 316 daltons.

### DEFINITIONS

As used herein, by “cleavage of an MvfR protein” is meant the separation of an MvfR protein into two or more peptides. “Cleavage of an MvfR protein” may be the result of a direct or indirect interaction of MvfR and one or more polypeptides or small chemical molecules, such as autoinducers. In addition, “cleavage of an MvfR protein” may result in its inactivation or change of function and may occur, for example, between amino acids 146 and 147 of the P. aeruginosa strain PA14 MvfR protein. Furthermore, “cleavage of an MvfR protein” may result in the generation of a polypeptide fragment that has a molecular weight of approximately 22 kDa.

As used herein, by “modification of an MvfR protein” is meant a post-translational modification of MvfR, or of a fragment thereof. Examples of such post-translational modifications include cleavage, glycosylation, and phosphorylation. However, a compound may also physically interact with MvfR and thereby modify or inactivate the protein. An MvfR protein may be modified, for example, by one of the compounds described herein that is present in the supernatant of a culture of P. aeruginosa strain PA14, but not in the supernatant of a culture of P. aeruginosa having an mvfR mutation, during the late stationary phase.

An “anti-fungal compound” of the invention may be a compound present in the supernatant of a culture of P. aeruginosa strain PA14, but not in the supernatant of a culture of P. aeruginosa having an mvfR mutation, at late stationary phase. In addition, such an anti-fungal compound may be present in the organic fraction of the supernatant of a culture of P. aeruginosa strain PA14, but not in the organic fraction of the supernatant of a culture of P. aeruginosa having an mvfR mutation, at late stationary phase. Alternatively, an anti-fungal compound may not be secreted into the supernatant, but may require the activity of MvfR. In this case, the presence of the compound may be directly dependent on the modification of MvfR. An anti-fungal compound may be used to treat a fungal infection of an animal, for example, a human. Examples of fungal infections include yeast infections, e.g., infection by Candida albicans or Saccharomyces cerevisiae, and Cryptococcus neoformans or Fusarium oxysporum infections. In addition, an anti-fungal compound may be used to treat an infection of a plant by a fungal pathogen (e.g., Fusarium oxysporum).

As used herein, by “autoinducer” is meant a diffusible small chemical molecule (e.g., one that is more soluble in ethyl acetate than in water) or peptide that is involved in the regulation or repression, either directly or indirectly, of virulence related target genes, such as lasB, lasA, apr, toxA, rhlAB, mvfR, hcnABC, phzA1B1C1D1E1F1G1, and phzA2B2C2D2E2F2G2. For example, an autoinducer may be an acyl-homoserine lactone, such as N-(3-oxododecanoyl)-L-homoserine lactone (C₁₂-HSL), N-butyryl-L-homoserine lactone (C₄-HSL), or the signal molecule 2-heptyl-3-hydroxy-4-quinolone (PQS), diketopiperazines (DKPs), or a fatty acid methyl ester. However, isomers and structural analogs of known autoinducers, such as 2-hydroxy-3-heptyl-4-quinolone and 2-heptyl-4-hydroxy-quinolone-N-oxide (described in Pesci et al. (Proc. Natl. Acad. Sci. USA 96:11229-11234, 1999)) are also encompassed by this definition.

In addition, an “autoinducer” may be a novel, or partially uncharacterized, small chemical molecule that is more soluble in ethyl acetate than in water, or a small peptide that is soluble in ethyl acetate. Furthermore, isomers of novel, or partially uncharacterized, small chemical molecule autoinducer, e.g., a D or L isomer, or analogs thereof, are included in this definition.

As used herein, by “more soluble in ethyl acetate than in water” is meant a compound which is at least 2, 3, 4, 5, 7, 10, 25, 50, or 100-fold more soluble in ethyl acetate than in water.

As used herein, by an “organic solvent” is meant a carbon-containing, non-aqueous compound in its liquid state, for example, ethyl acetate, chloroform, dimethyl sulfoxide, or an alcohol.

As used herein, by a “greater level” is meant an amount that is at least 20%, 30%, 50%, 75%, 90%, or 100% greater than the amount to which it is being compared. However, a “greater level” may also refer to an amount that is at least 2, 3, 5, 10, 50, 100, 500, or 1000-fold greater than the amount to which it is being compared.

As used herein, by “exponential phase” is meant the logarithmic phase of the bacterial growth curve where the bacteria are dividing at their maximal rate and where the overall number of bacteria in the growth medium continues to increase over time. The OD₆₀₀ (optical density) at which a bacterial strain enters into the exponential growth phase depends on the culture conditions, as well as on the size of the bacterium. However, one skilled in the art of microbiology can readily determine this time-point using standard techniques. For example Pseudomonas aeruginosa strain PA14 grown at 37° C. in Luria-Bertani medium (LB) is in the exponential growth phase when the OD₆₀₀=1.0.

As used herein, by “late exponential phase” is meant the time period immediately preceding the stationary phase of the bacterial growth curve. For example, Pseudomonas aeruginosa strain PA14 grown at 37° C. in LB is in the late exponential growth phase when the OD₆₀₀ is between 2.4 and 3.5.

As used herein, by “stationary phase” is meant the phase of the bacterial growth curve, immediately following the late exponential phase, at which there is no net gain or loss in the number of bacteria present in the growth medium. For example, Pseudomonas aeruginosa strain PA14 grown at 37° C. in LB is in the stationary phase when the OD₆₀₀>4.0, e.g., an OD₆₀₀ of 4.5.

By “purified” is meant separated from other components that naturally accompany it. Typically, a factor is substantially pure when it is at least 50%, by weight, free from proteins, antibodies, and naturally-occurring organic molecules with which it is naturally associated. Preferably, the factor is at least 75%, more preferably, at least 90%, and most preferably, at least 99%, by weight, pure. A substantially pure factor may be obtained by chemical synthesis, separation of the factor from natural sources, or production of the factor in a recombinant host cell that does not naturally produce the factor. Proteins, vesicles, and organelles may be purified by one skilled in the art using standard techniques, such as those described by Ausubel et al. (Current Protocols in Molecular Biology, John Wiley & Sons, New York, 2000). The factor is preferably at least 2, 5, or 10-times as pure as the starting material, as measured using polyacrylamide gel electrophoresis, column chromatography, optical density, HPLC analysis, or Western analysis (Ausubel et al., supra). Preferred methods of purification include immunoprecipitation, column chromatography such as immunoaffinity chromatography, magnetic bead immunoaffinity purification, and panning with a plate-bound antibody.

By “mutation” is meant an alteration in a naturally-occurring or reference nucleic acid sequence, such as an insertion, deletion, frameshift mutation, silent mutation, nonsense mutation, or missense mutation. Preferably, the amino acid sequence encoded by the nucleic acid sequence has at least one amino acid alteration from a naturally-occurring sequence. Examples of recombinant DNA techniques for altering the genomic sequence of a cell, embryo, fetus, or mammal include inserting a DNA sequence from another organism (e.g., a human) into the genome, deleting one or more DNA sequences, and introducing one or more base mutations (e.g., site-directed or random mutations) into a target DNA sequence.

By an “MvfR protein” or an “MvfR polypeptide” is meant having an amino acid sequence that is at least 20, 30, 40, 50, 60, 70, 80, 90, 95, 98, or 100% identical to at least 50, 100, 200, 250, or 300 amino acids of a Pseudomonas aeruginosa strain PA14 MvfR amino acid sequence, for example that shown in FIG. 10 or in GenBank Accession No. AF031571. Such an “MvfR protein” or an “MvfR polypeptide” may be found in various bacterial species including, for example, Salmonella typhimurium, Salmonella enterica, Escherichia coli, Azospirillum brasilense, and Agrobacterium tumefaciens.

Furthermore, an “MvfR protein” or an “MvfR polypeptide” may have an amino acid sequence that is at least 20, 30, 40, 50, 60, 70, 80, 90, 95, 98, or 100% identical to amino acids 1-65, 100-173, 196-206, and/or 227-253 of the sequence shown in FIG. 10 (SEQ ID NO:2).

By an “mvfR nucleic acid sequence” is meant a sequence that is at least 30, 40, 50, 60, 70, 80, 90, or 100% identical to that of the P. aeruginosa strain PA14 mvfR nucleic acid sequence, for example, the mvfR nucleic acid sequence spanning nucleotides 1,458 to 436 of the sequence shown in FIG. 11 (SEQ ID NO:3) or that of GenBank Accession number AF031571. In addition, an “mvfR nucleic acid sequence” may be present in a variety of bacterial species including, Salmonella typhimurium, Salmonella enterica, Escherichia coli, Azospirillum brasilense, and Agrobacterium tumefaciens.

By “substantially identical” is meant a polypeptide or nucleic acid sequence exhibiting at least 50%, preferably 60%, 70%, 75%, or 80%, more preferably 85%, 90% or 95%, and most preferably 99% identity to a reference amino acid or nucleic acid sequence. For polypeptides, the length of comparison sequences will generally be at least 15 contiguous amino acids, preferably at least 20 contiguous amino acids, more preferably at least 25, 50, 75, 90, 100, 150, 200, 250, or 300 contiguous amino acids, and most preferably the full-length amino acid sequence. For nucleic acids, the length of comparison sequences will generally be at least 45 contiguous nucleotides, preferably at least 60 contiguous nucleotides, more preferably at least 75, 150, 225, 275, 300, 450, 600, 750, or 900 contiguous nucleotides, and most preferably the full-length nucleotide sequence.

Sequence identity is typically measured using sequence analysis software with the default parameters specified therein (e.g., Sequence Analysis Software Package of the Genetics Computer Group, University of Wisconsin Biotechnology Center, 1710 University Avenue, Madison, Wis. 53705). This software program matches similar sequences by assigning degrees of homology to various substitutions, deletions, and other modifications.

By “high stringency hybridization conditions” is meant, for example, hybridization at approximately 42° C. in about 50% formamide, 0.1 mg/ml sheared salmon sperm DNA, 1% SDS, 2×SSC, 10% Dextran sulfate, a first wash at approximately 65° C. in about 2×SSC, 1% SDS, followed by a second wash at approximately 65° C. in about 0.1×SSC. Alternatively, “high stringency hybridization conditions” may include hybridization at approximately 42° C. in about 50% formamide, 0.1 mg/ml sheared salmon sperm DNA, 0.5% SDS, 5×SSPE, 1×Denhardt's, followed by two washes at room temperature in 2×SSC, 0.1% SDS, and two washes at between 55-60° C. in 0.2×SSC, 0.1% SDS.

Other features and advantages of the invention will be apparent from the following detailed description and from the claims.

### BRIEF DESCRIPTION OF THE DRAWINGS

FIG. 1 is a schematic representation of the relative positions of the pho34B12 locus and phnAB operon on the chromosome of P. aeruginosa. “ORF 1” and “ORF2” are the two open reading frames identified in the pho34B12 locus, where ORF2 is the mvfR gene, and “TnphoA” indicates the position of the TnphoA insertion in the pho34B12 locus.

FIG. 2 is a protein gel stained with Coomassie Blue to show exoproteins isolated from the wild-type P. aeruginosa PA14 strain (Lane 1) and from the mutant P. aeruginosa PA14 strains, pho34B12 (Lane 2), ORF1* (Lane 3), and ORF2* (mvfR mutant) (Lane 4). Extracellular proteins from the wild-type and mutant strains grown in LB media (OD₆₀₀=2.5-3.0) were isolated, separated on a 4-20% gradient polyacrylamide gel and stained with Coomassie Brilliant Blue R-250.

FIG. 3 is a series of two bar graphs showing the expression of mvfR-lacZ, lasR-lacZ, and rhlR-lacZ. Panel A shows the expression of an mvfR-lacZ transcriptional fusion in wild-type PAO1 and the following mutant strains: lasR⁻, rhlR⁻, and lasR⁻rhlR⁻. A plasmid containing an mvfR-lacZ transcriptional fusion gene was introduced into strains PAO1 (1), lasR⁻ (2), rhlR⁻ (3), and lasR⁻rhlR⁻ (4). The β-galactosidase activity was measured in these strains at OD₆₀₀=2.5-3.0. Panel B shows the expression of lasR-lacZ and rhlR-lacZ in the wild-type Pseudomonas aeruginosa PA14 strain (1) and in the Pseudomonas aeruginosa PA14 strain containing a mutation in ORF2 (ORF2*; the mvfR mutant) (2). Plasmids containing either lasR-lacZ or rhlR-lacZ were introduced into the wild-type and the ORF2* mutant Pseudomonas aeruginosa PA14 strains. The β-galactosidase activity was measured in these strains at OD₆₀₀=2.5-3.0.

FIG. 4 is a graph showing that the expression of the transcriptional fusion phnAB-lacZ is positively regulated by mvfR. Plasmids containing the phnAB-lacZ transcriptional fusion were introduced into both the wild-type Pseudomonas aeruginosa strain PA14 (1) and the ORF2* mutant of Pseudomonas aeruginosa strain PA14 (2), and the β-galactosidase activity was measured at OD₆₀₀=2.5-3.0.

FIG. 5 is a gel shift assay showing that the MvfR protein binds specifically to the promoter of the phnAB operon. Lane 1 only contains radio-labeled P1 (a 51-bp sequence 185-bp upstream of the start codon of phnAB operon); Lane 2 was left blank; Lanes 3-8 contain MvfR, radio-labeled P1, and X-fold non-radio-labeled P1, where, in Lane 3 X=0, in Lane 4 X=10, in Lane 5 X=20, in Lane 6 X=40, in Lane 7 X=80, and in Lane 8 X=160. Lane 9 was left blank. Lanes 10-12 contain MvfR, radio-labeled P1, and Y-fold non-radio-labeled P2 (a 51-bp sequence 460-bp upstream of the start codon of phnA/B operon), where, in Lane 10 Y=20, in Lane 11 Y-40, and in Lane 12 Y=80. Lane 13 contains MvfR, radio-labeled P1, and an anti-MvfR polyclonal antibody.

FIG. 6 is a series of Western blots showing the sub-cellular localization of the MvfR protein, and a graph showing the expression of mvfR and phnAB, at different growth phases. Plasmids containing the nucleic acid sequence encoding the MvfR-GST translational fusion were introduced into the P. aeruginosa PA14 strain. Protein extracts from cell fractions obtained from both PA14 and transformed strains grown to the indicated cell density were prepared, separated on a 10% polyacrylamide gel, and blotted onto IMMOBILON-P (PVDF) membranes. A monoclonal antibody against GST was used to detect the MvfR-GST fusion. The numbers to the right of each panel indicate the cell density (OD₆₀₀). In this Figure, “WT” is used to indicate the wild-type P. aeruginosa PA14 strain; “GST” is used to indicate the P. aeruginosa PA14 strain containing the MvfR-GST translational fusion; “P” stands for periplasmic; “C” stands for inner membrane; “S” stands for secreted; “M” stands for membrane; “I” stands for cytoplasmic membrane; and “OM” stands for outer membrane. The graph in Panel B shows the expression of mvfR and phnAB at different growth phases. The β-galactosidase activities in P. aeruginosa PA14 strains containing either an mvfR-lacZ or a phnAB-lacZ transcriptional fusion were measured at the growth phases indicated on the graph.

FIG. 7 is a series of Western blots showing the translocation and cleavage of MvfR in response to extracellular signals from wild-type and mutant P. aeruginosa PA14 strains. Protein preparations from the supernatant and the membrane fractions of untreated (Panel A), treated P. aeruginosa PA14 wild-type (Panel B), and ORF2* mutant (Panel C) cells were separated on a 10% polyacrylamide gel and blotted onto IMMOBILON-P (PVDF) membranes. A monoclonal antibody against GST was used to detect the MvfR-GST fusion protein. Both the wild-type Pseudomonas aeruginosa PA14 strain and the Pseudomonas aeruginosa PA14 strain containing an MvfR-GST translational fusion were grown in LB media until the OD₅₀₀ reached 2.5-3.0. The cells then were harvested and treated for 1 hour with cell-free cultures of wild-type and ORF2* mutant Pseudomonas aeruginosa PA14 strains grown to late stationary phase (OD₆₀₀>5.0).

FIG. 8 is a schematic diagram of a non-limiting model of how MvfR may function as an auto-regulated and membrane-associated transcription regulator of pyocyanin and exoprotein production. The early exponential phase is depicted in Panel I; the late exponential phase is depicted in Panels II and III; and the late stationary phase is depicted in Panel IV. In addition, “O” is used to refer to the outer membrane and “C” is used to refer to the cytoplasmic membrane.

FIG. 9 is the ORF1 amino acid sequence (SEQ ID NO:1).

FIG. 10 is the ORF2 (MvfR) amino acid sequence (SEQ ID NO:2).

FIG. 11 is the nucleic acid sequence corresponding to the pho34B12 locus containing ORF1 and ORF2 (SEQ ID NO:3). ORF1 spans nucleotides 361 to 1509 of this sequence and ORF2, which is transcribed in the opposite direction, spans nucleotides 1458 to 436 of this sequence. Accordingly, the overlap of these two open reading frames spans nucleotides 436 to 1458.

FIG. 12 is a series of HPLC-MS chromatographs. Panel A is the spectrum obtained from the ORF2 (mvfR) mutant P. aeruginosa strain PA14, panel B is the spectrum obtained from the ORF1 mutant P. aeruginosa strain PA14, and panel C is the spectrum obtained from the wild-type P. aeruginosa strain PA14. Five peaks (peaks 2-6) are missing in panel A that are present in the other two panels, indicating that at least five compounds are absent in the ORF2 (mvfR) mutants. Three of these compounds represent peaks at 9.18, 9.59, and 9.85 minutes of panel C, respectively, and two are likely to be 2-heptyl-3-hydroxy-4-quinolone (peak at 8.58 minutes of panel C) and N-(3-oxododecanoyl)-L-homoserine lactone (peak at 10.25 minutes of panel C). In addition, one peak is present in the ORF2 (mvfR) mutant (peak 1 at 4.79 minutes of panel A), that is absent in the wild-type and ORF1 mutant strains.

FIG. 13 is an HPLC-MS chromatogram from the wild-type P. aeruginosa strain PA14.

FIG. 14 is a series of mass spectra of the five peaks present in the wild-type P. aeruginosa strain PA14, but absent in the ORF2 (mvfR) mutant.

FIG. 15 is an HPLC-MS chromatogram from the ORF 1 mutant P. aeruginosa strain PA14.

FIG. 16 is a series of mass spectra of the five peaks present in the ORF1 mutant P. aeruginosa strain PA14, but absent in the ORF2 (mvfR) mutant.

FIG. 17 is an HPLC-MS chromatogram from the ORF2 (mvfR) mutant P. aeruginosa strain PA14.

FIG. 18 is a series of mass spectra from the ORF2 (mvfR) mutant P. aeruginosa strain PA14.

FIG. 19 is a schematic illustration of the domain organization of MvfR (SEQ ID NO:2). SP denotes Signal Peptide (1-33); HTH, helix-turn-helix signature (6-65); and LysR substrate, LysR substrate binding domain (156-293). The arrow indicates the cleavage site between residues 146-147.

FIG. 20 is a set of HPLC-MS chromatograms of the extracts of supernatants of PA14 (bottom), mvfR (middle), and phnAB (top) mutant. The peaks that correspond to peaks 2-6 of FIG. 13 are indicated.

FIG. 21 is a schematic illustration of the synthesis pathway used to synthesize the Pseudomonas quinolone signal compound.

FIG. 22 is a graph of the production of PQS (▴) by a P. aeruginosa culture as a function of time. Cell growth (♦) is measured as the optical density at 600 nm.

FIG. 23 is a set of (A) Unlabeled PQS and (B) PQS spectrum obtained after 15 hours of incubation in presence of the labelled putative PQS precursor.

FIG. 24 is a TIC chromatogram of a 24 hour P. aeruginosa culture extract. Numbers correspond to the ink of pseudomolecular ions and those in parentheses are their relative intensities.

FIG. 25 is an HPLC chromatogram indicating the twelve recovered fractions that were tested for anti-infective activity.

### DETAILED DESCRIPTION

One of the mechanisms utilized by P. aeruginosa to overcome the host defense system is the production of a large number of extracellular products, such as proteases, toxins, and lipases. The production of such extracellular products is controlled in a cell density-dependent manner using small diffusible signaling molecules, a process known as “quorum sensing” (QS) (Fuqua et al., Annu. Rev. Microbiol. 50:727-751, 1996; Fuqua et al., Annu. Rev. Genet. 35:439-468, 2001). The ability to coordinate gene expression is considered advantageous because it allows bacteria to appropriately “schedule” a concerted attack when sufficient cells are present to overwhelm the host defense response (De Kievit and Iglewski, Infect. Immun. 68:4839-4849, 2000). QS relies on the activation of specific transcriptional regulators by their corresponding autoinducers, which function as intercellular signals. In P. aeruginosa, as in most Gram-negative bacteria, these autoinducers are N-acyl-L-homoserine lactones (AHLs) (Pesci and Iglewski, Quorum sensing in Pseudomonas aeruginosa. In Cell-Cell Signaling in Bacteria, pp. 147-155. Edited by G. M. Dunny & S. C. Winans. Washington, D.C.: American Society for Microbiology, 1999). At least two separate QS systems (termed las and rhl), each of which consist of an AHL synthase (LasI or RhlI) and a cognate transcriptional regulator (LasR or RhlR), modulate gene transcription in response to increasing AHL concentrations in P. aeruginosa (Pearson et al., J. Bacteriol. 179:5756-5767, 1997; Pesci et al., J. Bacteriol. 179:3127-3132, 1997; Passador et al., Science 260:1127-1130, 1993; Ochsner and Reiser, Proc. Natl. Acad. Sci. USA 92:6424-6428, 1995; Latifi et al., Mol. Microbiol. 17:333-343, 1995; Winson et al., Proc. Natl. Acad. Sci. USA 92:9427-9431, 1995). The major products of lasI and rhlI are N-(3-oxododecanoyl)-L-homoserine lactone (3-oxo-C₁₂-HSL) and N-butyryl-L-homoserine lactone (C₄-HSL), respectively (Pearson et al., Proc. Natl. Acad. Sci. USA 92:1490-1494, 1995; Pearson et al., J. Bacteriol. 179:5756-5767, 1997). Together, these two systems comprise a hierarchical cascade (where the las system regulates the rhl system) that coordinates the expression of numerous genes as well as the production of additional QS signals (Latifi et al., Mol. Microbiol. 21:1137-1146, 1996; Pesci et al., J. Bacteriol. 179:3127-3132, 1997). More recently, a third intercellular signaling molecule, the Pseudomonas quinolone signal (PQS), which is not involved in sensing population density, but is related to the QS hierarchy and upregulates the rhl system, has been identified (Pesci et al., Proc. Natl. Acad. Sci. USA 96:11229-11234, 1999; McKnight et al., J. Bacteriol. 182:2702-2708, 2000).

Further illustrating the complexity of this system, an increasing number of genes found to be involved in the regulation of the whole QS cascade are being reported. qscR, a gene coding for a homologue of LasR and RhlR, was recently identified (Chugani et al., Proc. Natl. Acad. Sci. USA 98:2752-2757, 2001). This gene governs the timing of QS-controlled gene expression and acts as a repressor of lasI. Whether QscR requires binding of a cognate autoinducer molecule is unknown. RsmA, the P. aeruginosa homologue of the E. coli CsrA protein, was shown to act as a global posttranscriptional regulator of secondary metabolites, by modulating the QS circuitry (Pessi et al., J. Bacteriol. 183:6676-6683, 2001), and the P. aeruginosa DksA homologue inhibits the expression of rhlI by an undetermined mechanism (Branny et al., J. Bacteriol. 183:1531-1539, 2001). These regulatory steps provide overall coordination of QS and temporal gene expression in response to cell-to-cell communication. The QS system thus appears to constitute a global regulatory system in P. aeruginosa. In fact, it is estimated that up to 4% of P. aeruginosa genes are regulated by QS (Whiteley et al., Proc. Natl. Acad. Sci. USA 96:13904-13909, 1999).

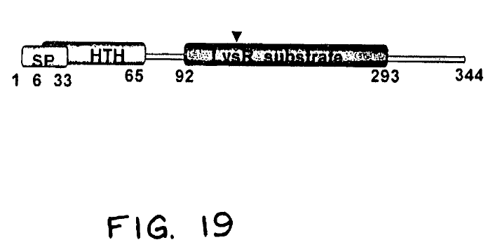

Previous studies showed that mutations in the TnphoA-induced pho34B12 locus lead to the reduced ability of P. aeruginosa strain PA14 to cause disease in plants and animals, and affect the production of virulence-associated factors relevant in mammalian pathogenesis (Ralune et al., Proc. Natl. Acad. Sci. USA 94:13245-13250, 1997). As is described in more detail below, we identified a novel QS-related transcriptional activator, MvfR, which is required for the production of intercellular signal molecules as well as pyocyanin, elastase, phospholipase and a large number of unidentified secreted products in P. aeruginosa. We created a non-sense mutation in the pho34B12 locus and hereafter we will refer to this locus as mvfR. Our studies have shown that a mutation at this locus leads to the reduced ability of P. aeruginosa strain PA14 to cause disease in plants, nematodes, insects, and mice. Mutant mvfR caused death in 35% of the burned mice as compared to wild-type strain PA14, which caused at least 80-90% mortality. Bioinformatics analysis indicates that the predicted protein encoded by mvfR belongs to the LysR family of bacterial transcriptional regulators (FIG. 19). This class of proteins, includes bacterial gene activator proteins which control the expression of genes associated with a multitude of highly diverse cellular processes, ranging from amino acid biosynthesis, CO₂ fixation, ion transport, antibiotic resistance, initiation of nodulation, chromosomal replication and control of virulence. It has been shown that at least some members of this family act either as tetramers or dimers of identical polypeptides, 270-330 amino acid residues in length. The members of this family share stretches of sequence similarity over approximately 270 residues, with the highest degree of conservation in the 66 N-terminal residues. This portion of the polypeptide includes a likely helix-turn-helix motif believed to play a role in DNA binding. Furthermore, family members contain a LysR substrate-binding domain towards their C-terminus. This domain is involved in co-inducer recognition and/or response, and is required for transcriptional activation. Moreover, the LysR substrate-binding domain has been shown in some studies to bind small molecules.

In the following examples, we show that the expression of mvfR is cell density-dependent and peaks at the late exponential phase. Our data also indicate that the MvfR protein is associated with the cytoplasmic membrane and that it is cleaved when cells reach stationary phase. Compounds secreted at high levels by P. aeruginosa strain PA14 during the stationary phase may promote the cleavage of MvfR, thereby inactivating the protein. However, these compounds may also inactivate MvfR or inhibit virulence by another mechanism. Furthermore, the signal(s) for the modification of the MvfR protein likely is controlled by the mvfR gene itself.

In addition, prior work indicates that pyocyanin, a blue-green pigmented phenazine, may contribute to the persistence of P. aeruginosa in the lungs of cystic fibrosis patients, since quantities of this phenazine capable of altering eukaryotic cell function could be isolated from the sputum of these patients (Wilson et al., Infect. Immun. 56:2515-2517, 1988). Various in vitro studies using purified pyocyanin have shown that pyocyanin inhibits mammalian cell respiration, disrupts the beating of human cilia, inhibits the growth of epidermal cells, and inhibits the release of IL-2, which, in turn, leads to the inhibition of T-lymphocyte proliferation and immunoglobulin secretion by B-lymphocytes (Wilson et al., Infect. Immun. 56:2515-2517, 1988; Wilson et al., J. Clin. Invest. 79:221-229, 1987; Ulmer et al., Infect. Immun. 58:808-815, 1990).

Our studies demonstrated that mutations mapping to the pho34B12 locus completely abolish the production of pyocyanin (Rahme et al., Proc. Natl. Acad. Sci. USA 94:13245-13250, 1997). As is noted above, and described in more detail below, we have characterized MvfR, a protein encoded by a gene in the pho34B12 locus. MAR binds to the promoter region, and positively regulates the expression, of the phnAB operon, and controls the production of elastase, phospholipase, autoinducer I (3-oxo-dodecanoyl homoserine) (Pearson et al., Proc. Natl. Acad. Sci. USA 91:197-201, 1994), the recently identified autoinducer-like molecule, 2-heptyl-3-hydroxy-4-quinolone (Pseudomonas Quinolone Signal (PQS)) (Pesci et al., Proc. Natl. Acad. Sci. USA 96:11229-11234, 1999), as well as the expression level of various P. aeruginosa secreted proteins. Accordingly, the level of MvfR expression, in addition to the activity of the protein itself, is likely to affect the virulence of P. aeruginosa in both animal and plant hosts. As is described in the following non-limiting examples, we also show that a compound expressed at higher levels by P. aeruginosa strain PA14 during late exponential phase may promote the cleavage of MvfR or otherwise inactivate the protein. Such a compound is useful for treating, stabilizing, or preventing an infection by any bacterium, e.g., P. aeruginosa, that expresses an MvfR protein.

These examples are provided for the purpose of illustrating the invention and should not be construed as limiting.

### Example 1

### Analysis of the MvfR Protein

### The Mutation in ORF2 (mvfR) is Responsible for the Mutant Phenotype of phoA34B12

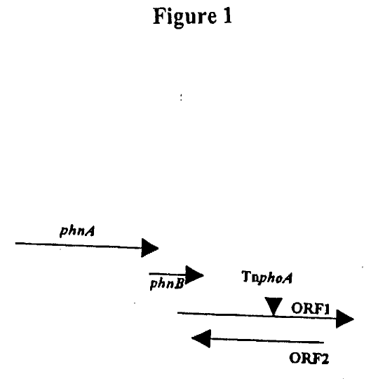

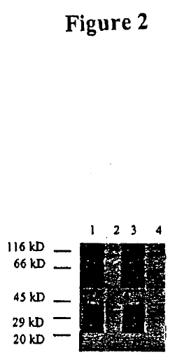

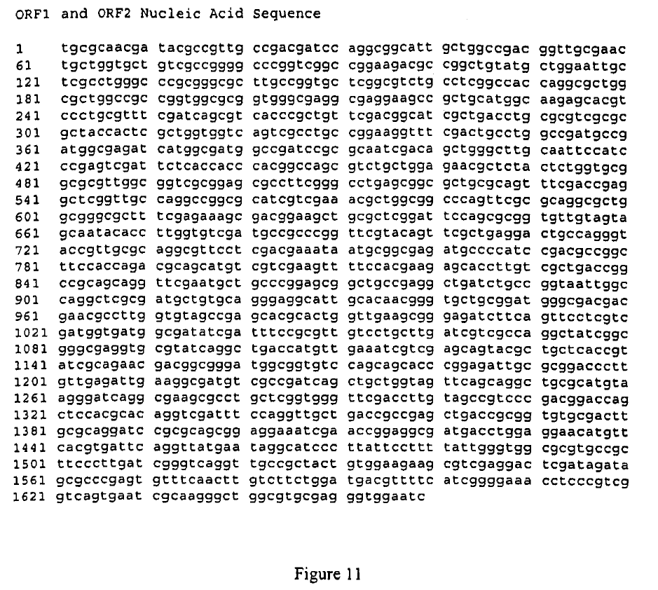

Previous studies identified two overlapping open reading frames (ORFs), in opposite orientations, in the pho34B12 locus (GenBank Accession No. AF031571; Rahme et al., Proc. Natl. Acad. Sci. USA 97:8815-8821, 1997; FIGS. 1 and 9-11). Using Northern blot analyses, we showed that both ORFs are transcribed. To determine the function of each ORF, we introduced a nonsense point mutation into each of the ORFs using polymerase chain reaction (PCR). We then marker-exchanged the mutant ORF into the chromosome of the wild-type P. aeruginosa strain PA14 by homologous recombination. Next, we analyzed the phenotypes of the resulting point mutants (designated as ORF1* and ORF2*, respectively). Both mutants were tested for virulence in plants and animals using an Arabidopsis leaf infiltration assay (Rahme et al., Science 268:1899-1902, 1995) and a mouse thermal injury model (Stevens et al., J. Burn Care and Rehabil. 15:232-235, 1994), respectively. The phenotypes of both point mutants were then compared with those of the pho34B12 mutant. As is summarized in Table 1, the mutation in ORF1 (ORF1*) does not affect the wild-type phenotype. In contrast, as in the mutant phoA34B12, the mutation in ORF2 (ORF2*; the mvfR mutant) results in an attenuated virulence phenotype in both plants and animals, which is demonstrated by an approximately 320-fold decrease in growth in Arabidopsis and a reduced mortality rate of 35% in mice, instead of the approximately 80-90% mortality rate observed with the wild-type P. aeruginosa strain PA14 (Table 1). In addition, analogous to what is observed in the pho34B12 mutant, the mutation in ORF2* results in a lack of pyocyanin production and in decreased levels of elastase and phospholipase. Furthermore, ORF2* mutant P. aeruginosa exhibit decreased levels of various secreted exoproteins (FIG. 2), which is similar to the decreased levels of secreted exoproteins seen in the mutant phoA34B12. The importance of the ORF2 (mvfR) locus in the quorum sensing cascade is further demonstrated by the fact that the ORF2* mutation results in decreased levels of the P. aeruginosa quorum-sensing signal molecules PAI I and PQS (Table 1).

TABLE 1
Phenotypic analyses of wild-type (W-T), or mutant (pho34B12,
ORF1*, and ORF2*) P. aeruginosa PA14 strains.

| | Growth in | Mortality in | | | Auto- |
|---|---|---|---|---|---|
| | Arabidopsis | Burn Mouse | | | Inducers |
| | (cfu/leaf | Model | Pyocy- | Secreted | (PAI I & |
| Strain | disc) | (5 × 10⁵ cfu) | anin | proteins | PQS) |
| W-T | 1.6 × 10⁷ | 90% | 100% | Normal | 100% |
| PA14 | | | | | |
| 34B12 | 6.8 × 10⁵ | 60% | <10% | Reduced | ~10% |
| ORF1* | 2.8 × 10⁷ | 90% | 120% | Normal | 100% |
| ORF2* | 5.0 × 10⁴ | 35% | <10% | Reduced | ~10% |

In Table 1, the numbers in the column labeled “Growth in Arabidopsis” were generated using two leaf discs from each of four samples for each strain and reflect bacterial growth four days post-infection. To generate the mortality rate in the “Burn Mouse Model,” eight to ten mice were used in each experiment.

In addition, we assayed the ability of P. aeruginosa PA14 and the mvfR mutant to kill eukaryotic unicellular cells using the yeast Saccharomyces cerevisiae. P. aeruginosa PA14 and yeast were cultivated overnight in LB medium at 37° C. and in YPD medium at 30° C., respectively. The surface of a YPD agar plate was then evenly covered with 100 μl of the S. cerevisiae culture. After one hour of drying, 5 μl drops of the P. aeruginosa cultures were deposited side by side directly on the yeast lawn. Finally, the plate was incubated for 24 hrs at 37° C. Analysis of the results on the following day showed that PA14 was able to grow on the plate and produce a clearing zone around the colony, indicating that it inhibited the growth of the yeast cells in the vicinity of the bacteria. In contrast, no clearing was observed around the colony formed by the mvfR mutant, indicating that an mvfR mutation reduces the anti-fungal activity of P. aeruginosa.

MvfR(ORF2) Functions Independently of Quorum Sensing Regulators lasR and rhlR.

Because site directed mutagenesis studies demonstrate the importance of the ORF2 locus (also referred to herein as mvfR, for multiple virulence factors regulator) in the pathogenesis of P. aeruginosa-induced infection, we focused our subsequent efforts on this gene.

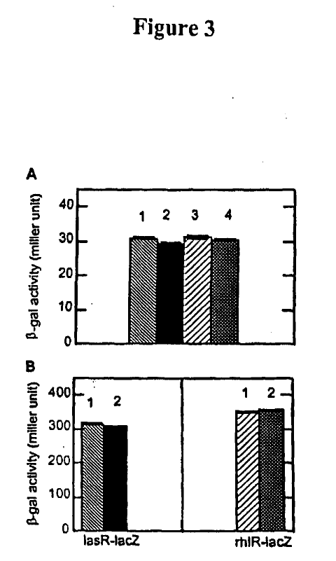

The phenotypic analysis of the ORF2* mutant indicates that the mvfR gene is regulating the production of autoinducers PAI I and PQS. We therefore performed experiments to determine the relationship between mvfR and the two known quorum sensing regulators of P. aeruginosa, lasR and rhlR. Using β-galactosidase transcriptional fusions of the mvfR, lasR and rhlR genes, we performed the following experiments to examine whether mvfR, lasR, and rhlR control or regulate each other's expression. In the first experiment, the expression of the mvfR-lacZ transcriptional fusion in the wild type P. aeruginosa strain PAO1, as well as in the PAO1 isogenic mutants lasR⁻, rhlR⁻, and lasR⁻rhlR⁻ (Pearson et al., J. Bacteriol. 179:5756-5767, 1997) was studied by measuring β-galactosidase activity. As is shown in FIG. 3A, β-galactosidase activity in the three mutant strains was not significantly different from that of the wild-type strain indicating that the expression of mvfR is not controlled by either of these two quorum sensing regulators. In the second experiment, the expression of lasR-lacZ and rhlR-lacZ transcriptional fusions in PA14 and in the ORF2* mutant strain was determined. The data shown in FIG. 3B reveal that mvfR does not regulate the expression of either of these two quorum sensing regulators, as is indicated by the β-galactosidase activities for both the lasR and the rhlR promoters being essentially the same in both wild-type and mutant strains. Accordingly, our results indicate that, although mvfR regulates quorum sensing-dependent components, mvfR does not regulate the known quorum sensing regulators at the level of transcription, nor is the transcription of mvfR controlled by these regulators.

mvfR Encodes a Transcriptional Regulator of the LysR Family that Positively Regulates the Expression of the phnAB Operon.

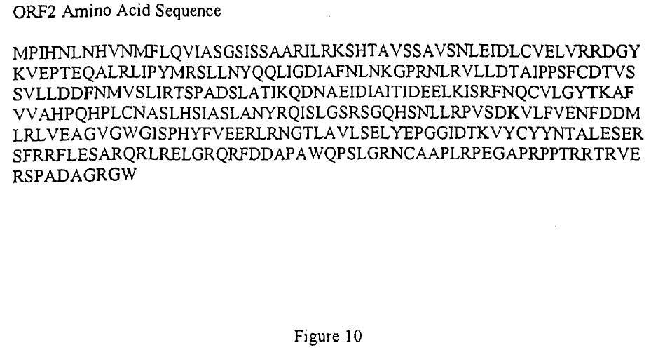

The fact that mvfR controls diverse pathogenicity functions makes it likely that mvfR encodes a regulatory molecule. This statement is consistent with the predicted protein sequence of MvfR that contains a helix-turn-helix (HTH) motif at the N-terminus (amino acids 6-65 of the sequence shown in FIG. 10), which bears a strong similarity to the conserved HTH signature motif belonging to the LysR family of transcription regulators (LTTRs) (Table 2; Schell, Ann. Rev. Microbiol. 47:597-626, 1993). In addition, MvfR contains domains involved in co-inducer response recognition and/or response (amino acids 100-173 and 196-206 of the sequence shown in FIG. 10), as well as a domain required for both DNA binding and co-inducer response (amino acids 227-253 of the sequence shown in FIG. 10).

TABLE 2
LTTRs containing helix-turn-helix motif are present in a variety of
bacteria.

| Accession | | | | |
|---|---|---|---|---|
| No. | Organism | Name | Function | Similarity |
| P37459 | Salmonella | Sinr Protein | Probable regulatory protein. Its | Belongs to the |
| | typhimurium | | target is not known. | LysR family of |
| | | | | transcriptional |
| | | | | regulators. |
| P24417 | Salmonella | Virulence genes | Positive regulator for the | Belongs to the |
| | enterica | transcriptional | plasmid-encoded virulence | LysR family of |
| | | activator | factors SPVA, SPVB, and SPVC | transcriptional |
| | | | | regulators. |
| P52044 | Escherichia coli | Hypothetical | — | Belongs to the |
| | | transcriptional | | LysR family of |
| | | regulator in | | transcriptional |
| | | SBM-FBA | | regulators. |
| | | intergenic region. | | |
| P52661 | Azospirillum | Galactose- | Activator of the expression of | Belongs to the |
| | brasilense | binding protein | sugar binding protein precursor | LysR family of |
| | | regulator (GBP- | (chvE) when bound to its | transcriptional |
| | | regulator). | inducer and represses its | regulators. |
| | | | expression in the absence of | |
| | | | inducer (L-arabinose, D-fuctose | |
| | | | or D-galactose) | |
| P52668 | Agrobacterium | PCA operon | Activates transcription of the | Belongs to the |
| | tumefaciens | transcriptional | PCADCHGB operon for the | LysR family of |
| | | activator. | catabolism of the phenolic | transcriptional |
| | | | compound protocatechuate. | regulators. |

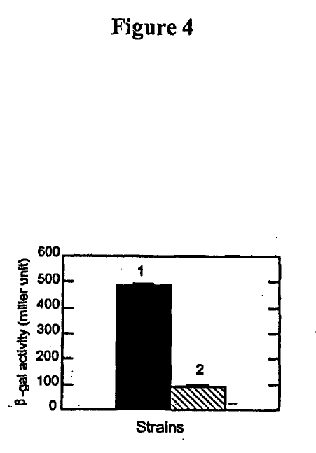

Given our data showing that a mutation in mvfR leads to a deficiency in pyocyanin, and since mvfR is located next to phnAB (FIG. 1), we wanted to characterize this operon further to determine if mvfR encodes a transcription factor. To this end, we constructed a plasmid containing the phnAB-lacZ transcriptional fusion and introduced it into both the wild-type and the ORF2* mutant strain PA14. The expression of lacZ was determined in the two strains by measuring their β-galactosidase activity. The results from this experiment showed that the β-galactosidase activity in the ORF2* strain is about 6-7 fold lower than that seen in the wild-type (FIG. 4). Accordingly, mvfR is likely to positively regulate the expression of the phnAB operon. RNA blot analysis of these two strains using the phnAB coding sequence as the nucleotide probe confirmed these results.

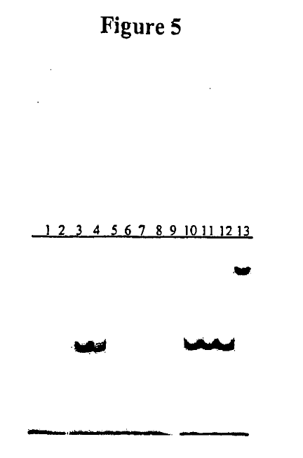

To determine whether the MvfR protein physically interacts with the promoter of the phnAB operon, a gel electrophoresis mobility shift assay was performed in which a 51 bp fragment (P1) about 185 bp upstream of the start codon of phnA was used as a DNA probe. The 51 bp fragment (P1) contains a consensus sequence found in the target genes' promoters with which the LysR-like transcription factors interact. Although the exact transcription start point in the phnAB operon has not been identified, we found a putative-10 bp-sequence about 120 bp upstream of the start codon of phnA. Another 51 bp fragment (P2), located at about 460 bp upstream of the start codon, was used for the competition binding assay. As is illustrated in FIG. 5, radio-labeled P1 was incubated in the absence (lane 1) and the presence (lane 3) of MvfR protein purified from E. coli. The appearance of a shifted band in lane 3 indicates that MvfR binds to P1. This interaction is specific because it can be competed away by excess non-radio-labeled P1 (FIG. 5, lanes 4-7), but not by an excess amount of P2 (FIG. 5, lanes 9-11). The observation that the shifted band (complex) contains MvfR protein is confirmed by the formation of a supershifted band after the addition of a polyclonal antibody raised against the C-terminus of MvfR to the binding reaction (FIG. 5, lane 13).

Cytoplasmic Membrane-Bound MvfR Protein is Cleaved when Cells Reach Stationary Phase and Modification of the MvfR Protein is Regulated by a Secreted Signal that Requires Functional MvfR

Previous studies showed that the product of the pho34B12 locus is either membrane-spanning or secreted (Rahme et al., Proc. Natl. Acad. Sci. USA 94:13245-13250, 1997). This conclusion was derived from the results of an assay using a P. aeruginosa UCBPP-PA14 strain containing an insertion of TnphoA at the pho34B12 locus, the phoA34B12 mutant, where a positive alkaline phosphatase activity indicates that the gene product of the gene into which the phoA gene is inserted is either secreted or otherwise exposed to the extracellular environment. Our subsequent examination of the orientation of the TnphoA insertion in the phoA34B12 mutant revealed that the phoA gene is in frame with mvfR. Moreover, a motif search performed on the MvfR sequence also identified a possible signal peptide at the extreme N-terminus of MvfR. Based on the phoA⁺ phenotype of the phoA34B12 mutant and the presence of a putative signal peptide, MvfR is likely to be a secreted protein.

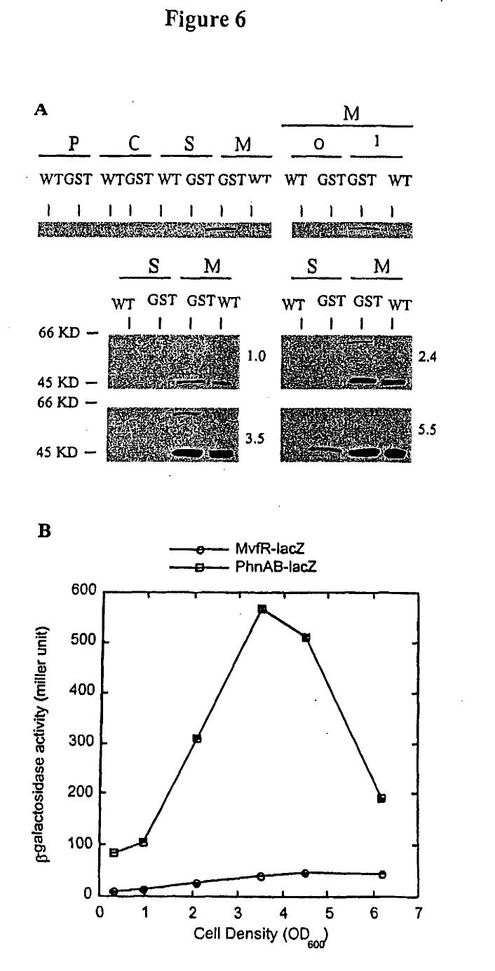

In addition, we wanted to determine the localization of the MvfR protein to see whether MvfR can function as a transcription factor that binds DNA. We generated a translational fusion protein in which the GST gene from Schistosoma japonicum was linked in frame to the C-terminus of MvfR, since the polyclonal antibody raised against a synthetic peptide at the C-terminus of MvfR was unable to produce an unambiguous signal in P. aeruginosa immunoblot studies. The plasmid containing the fusion protein was able to complement the pyocyanin-deficient phenotype of the ORF2* strain, confirming that the fusion protein functions like the endogenous MvfR protein. We then introduced the mvfR-GST fusion into the wild-type strain and designated the transformant PA14/GST. A monoclonal antibody against the GST protein was used to detect the fusion protein in the immunoblot assays. The wild-type strain lacking the fusion protein was used as a control in all the assays. We expected that the fusion protein band detected by the monoclonal antibody should be about 64 kD, the sum of MvfR (38 kD, as predicted) and GST (26 kD). As is shown in FIG. 6A, immunoblot assays were performed on fractionated cell extracts from different bacterial growth stages. No fusion protein was detected by the GST monoclonal antibody when cells are in the early exponential growth phase (i.e., OD₆₀₀-1.0); however, a protein band of about 64 kD, unique to the PA14/GST strain, was detected in the membrane fraction when cells reached the late exponential phase (i.e., between OD₆₀₀=2.4 and 3.5; FIG. 6A). The 64-kD band was detected in the cytoplasmic membrane fraction when the membrane fraction was further separated into cytoplasmic and outer membrane fractions. Moreover, when cells grew to the stationary phase (i.e., OD₆₀₀=5.5), the band of 64 kD in the membrane fraction diminished and an additional band of about 48 kD, unique for strain PA14/GST, was then detected in the supernatant (FIG. 6A). These data suggest that the expression of mvfR is cell density dependent and peaks at the late exponential phase. The results also show that the MvfR protein is associated with the cytoplasmic membrane and that it is cleaved when cells reach stationary phase.

Furthermore, we studied whether cleavage of the MvfR protein represents a way of regulating the activity of MvfR. To address this matter, we first examined the expression profile of the phnAB operon, which is under the regulation of mvfR. The β-galactosidase activities of the phnAB-lacZ transcriptional fusion were measured in PA14 to determine whether the expression profiles of the phnAB operon correlate with the expression and modification of the MvfR protein at different growth phases. As is indicated in FIG. 6B, the β-galactosidase activity generated by the phnAB-lacZ transcriptional fusion was low when cells were in early exponential phase, increased during exponential growth, and peaked in the late exponential phase. The β-galactosidase activity started to decrease when cells entered into stationary phase. This pattern of β-galactosidase activity is consistent with the profile of the mvfR expression and its modification.

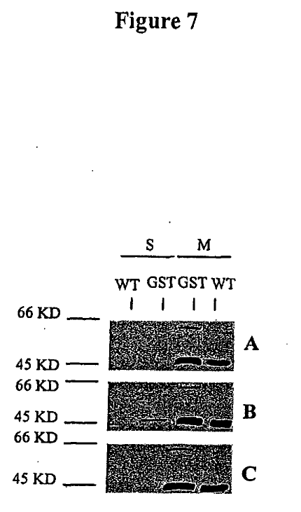

We also performed the following experiments to determine whether the modification of MvfR protein is regulated, and not simply due to cell lysis. In these experiments, we tested whether the modification of the MvfR protein can be triggered before cells reach the stationary phase. In the first experiment, both PA14 and PA14/GST strains were grown to late exponential phase (OD₆₀₀=2.5-3.0, when the expression of mvfR is maximal) and the cells were pelleted and re-suspended in cell-free supernatant obtained from the PA14 strain grown to stationary phase (OD₆₀₀>5.0). The cells were then incubated at 37° C. for one hour before the fractionated cell extracts were prepared. As is shown in FIG. 7, panel B, the band of 48 kD was already detected in the supernatant of PA14/GST strain in late exponential phase after treatment with the cell-free supernatant of PA14 from the stationary culture. In contrast, the 48 kD-band was absent in the supernatant of PA14/GST strain grown to the same stage in untreated LB media (FIG. 7, panel A). We performed a second experiment using a procedure similar to that used in the first experiment, except that we used cell-free supernatant obtained from a ORF2* stationary phase culture instead of from the wild-type strain (FIG. 7, panel C). In this experiment, the 48 kD-band was not detected in the extracellular fraction of PA14/GST cells treated with the ORF2* supernatant.

To demonstrate that the 48-kD fragment does not represent an experimental artifact due to our use of an anti-GST monoclonal antibody rather than an anti-MvfR antibody, we isolated the 48-kD fragment from the extracellular fraction and sequenced the first five amino acids of the N-terminus. We determined that the first five amino acids are identical to the amino acids corresponding to positions 147 to 151 of the MvfR amino acid sequence of FIG. 10. The predicted molecular weight of cleaved MvfR (amino acids 147-344; 22 kD) plus GST (26 kD) is 48 kD, matches the size of the fragment observed in the extracellular fraction. We also probed the cell fractions of PA14/GST grown to late exponential (OD₆₀₀=2.5-3.0) and stationary phase (OD₆₀₀>5.0) with an anti-secB (a cytoplasmic protein marker) antibody in immunoblot assays. The results of these experiments showed that secB was only present in the cytoplasmic fractions in both growth stages. In addition, we did not detect secB in the supernatant fractions. In view of the data presented in this section, we conclude that the cleavage of MvfR protein is a regulated process rather than a random consequence of cell lysis. Moreover, we show that the signal for the modification of MvfR protein is secreted and is controlled by the mvfR gene itself. Furthermore, the above results indicate that a signal is contained in the late stationary phase cell supernatants of PA14 that is likely to be responsible for the cleavage and release of the MvfR-GST fusion into the bacterial cell supernatant.

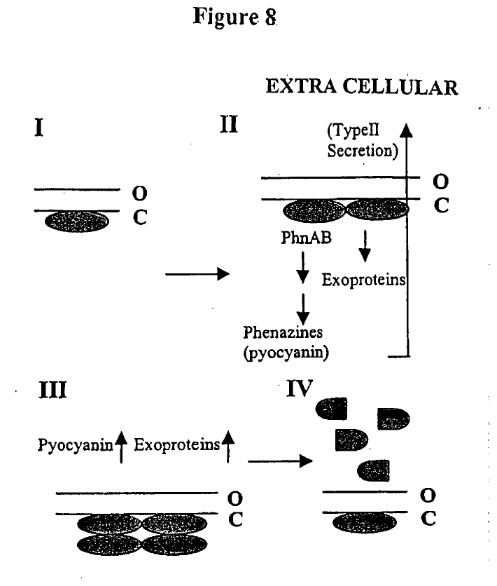

We also addressed whether cleavage of the MvfR protein results in its inactivation. To do so, we examined the effect of MAR modification on pyocyanin production. For this experiment, PA14/GST was grown overnight in LB medium and sub-cultured into either fresh LB medium alone, or into fresh LB medium to which the concentrated cell-free supernatant from wild-type PA14 grown to stationary phase was added in a 1:1 ratio (e.g., 10 ml of concentrated PA cell-free supernatant diluted in 10 ml LB medium). The amount of pyocyanin was measured at the time of sub-culturing and at different cell densities (OD₆₀₀=1.5, 2.5, and 4.4, respectively). The addition of the stationary phase cell-free supernatant from PA14 at the beginning of sub-culturing appears to completely abolish the production of pyocyanin (PA14+Sup; no increase of pyocyanin was observed along with cell growth) whereas plain LB medium does not exhibit this effect (PA14; pyocyanin level significantly increased during cell growth). Given that mvfR positively regulates pyocyanin production, the result of the above experiment indicates that modification of MvfR protein leads to inactivation or down-regulation of MvfR. A non-limiting model based on these results is shown in FIG. 8.

Chromatographic Analysis of the Cell Free Supernatant of PA14 and mvfR Mutant P. aeruginosa

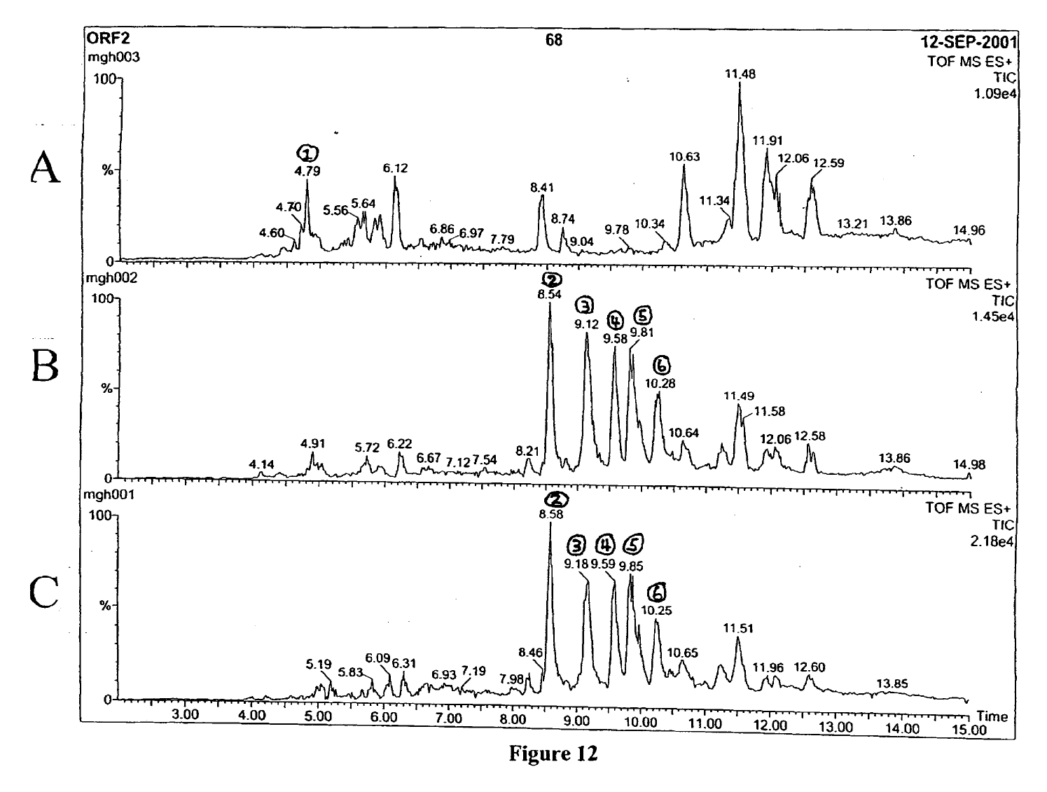

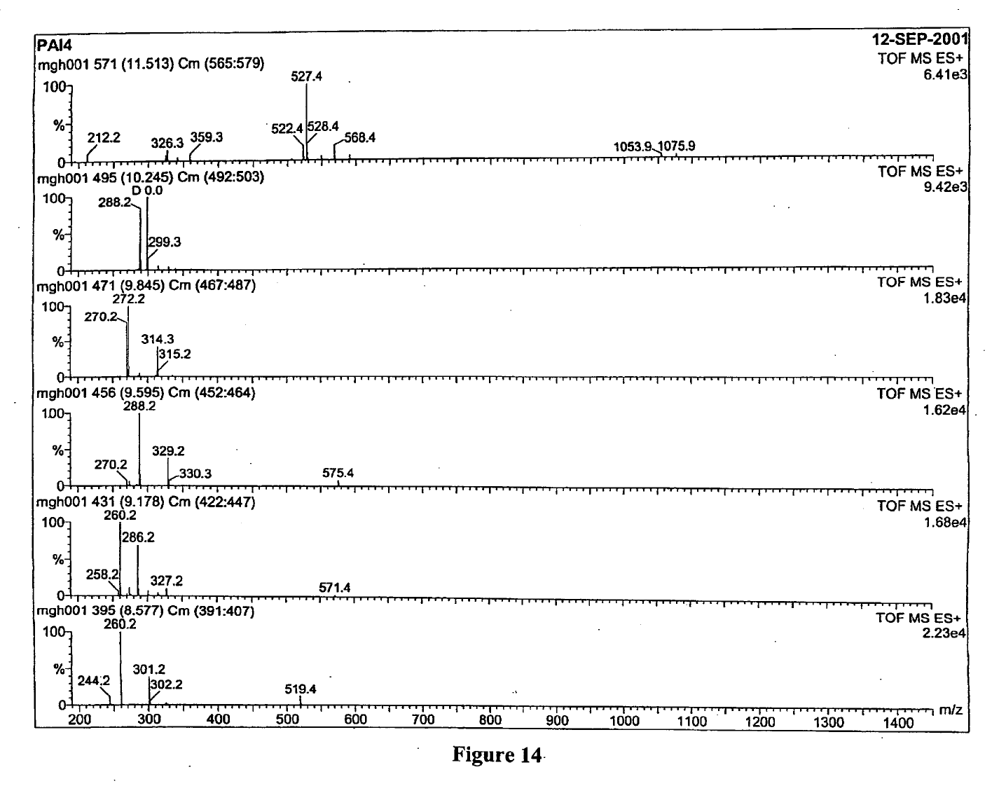

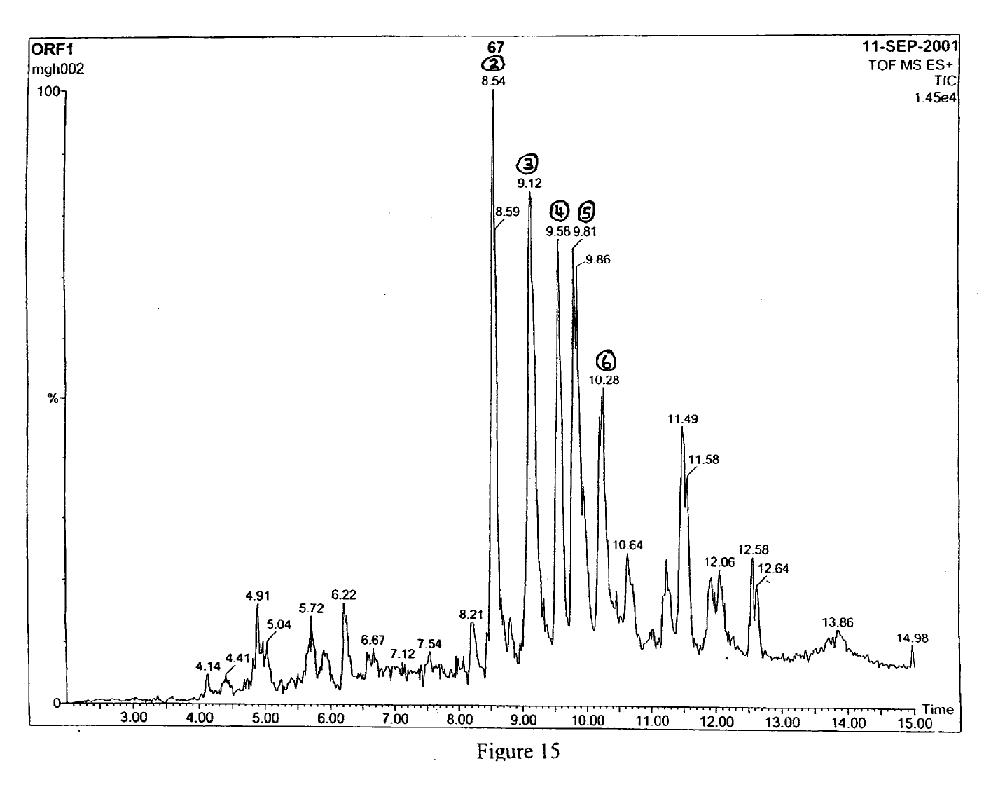

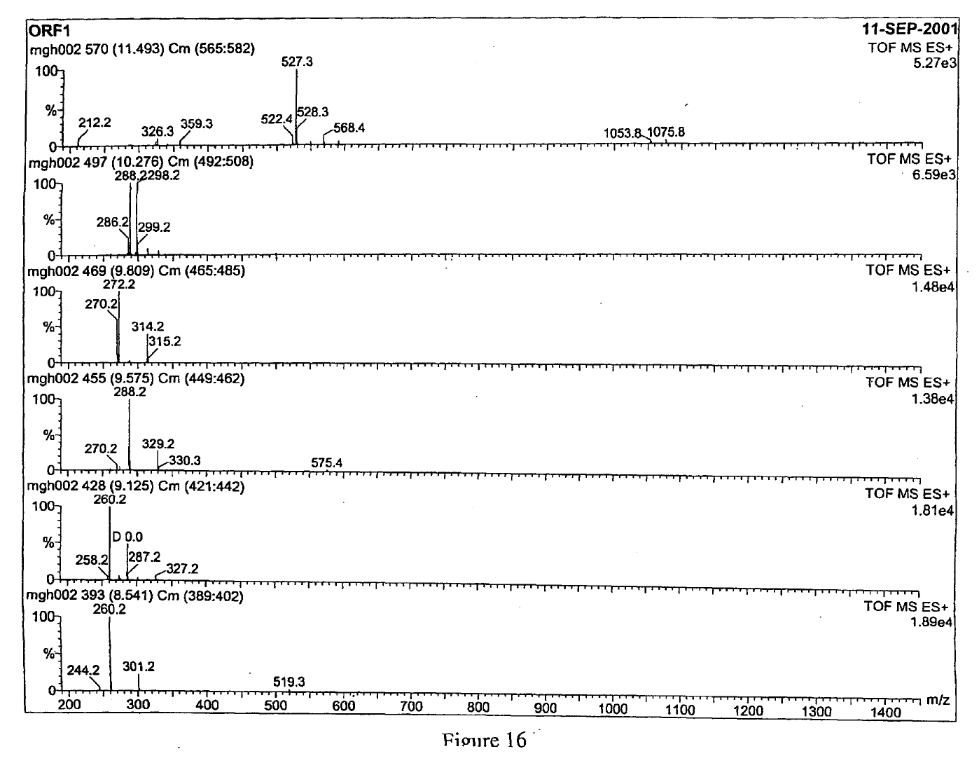

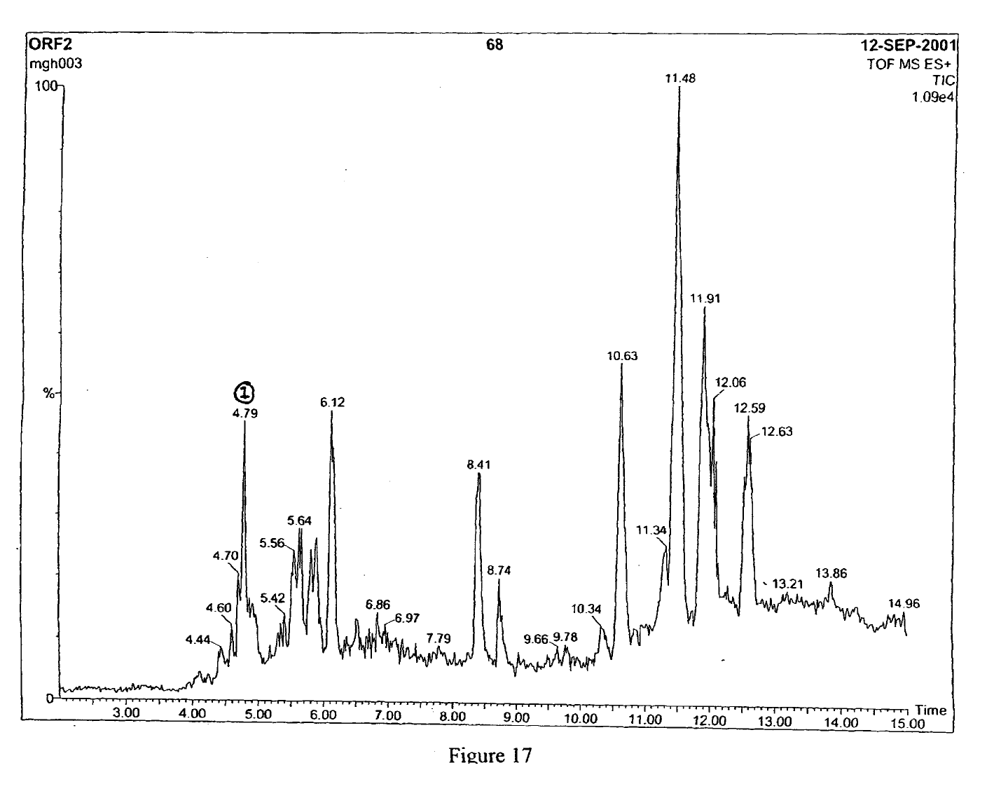

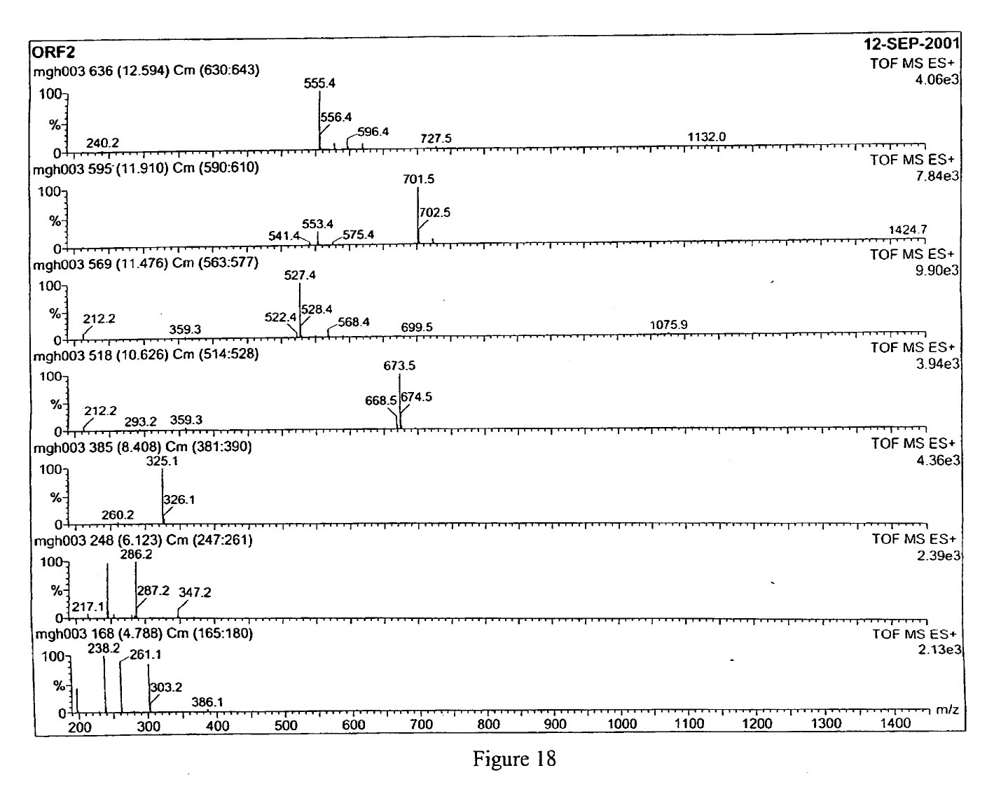

Liquid Chromatography (LC)/Mass Spectrometry (MS) analysis of the ethyl acetate extracts from wild-type PA14, ORF1* mutants, and the ORF2* mutants were compared (FIGS. 12-18). The results of this comparison suggest that the ORF2* mutant, which contains a nonsense mutation in the mvfR locus, likely lacks C₁₂-HSL and PQS. In addition, the level of several molecules described herein is also significantly reduced, and the level of one molecule is increased, in the ORF2* mutant (FIGS. 12-18 and Table 3). At least one of these molecules may be an autoinducer-like molecule because the molecules all co-purify with other autoinducers. Furthermore, the synthesis of these molecules is also under the regulation of MvfR transcriptional activator. Besides the molecules described above, the ORF2* mutant supernatant contains at least one peak that is only present in the mutant, and also may contain additional molecules that regulate MvfR cleavage.

All the above data strongly suggest that the known autoinducers (C₁₂-HSL, C₄-HSL, and PQS), as well as the molecules newly described herein, may be coordinately regulated and that they may control, either directly or indirectly, and possibly in combination with each other, the modification and release MvfR protein at late stationary phase. In addition, the molecules newly described herein may regulate the production of MvfR protein.

TABLE 3
LC/MS analysis of wild-type and ORF1* or ORF2*
mutant P. aeruginosa PA14 strains.

| Base | | | | | |
|---|---|---|---|---|---|
| peak ion | PA14 | | | Likely | Correspond- |
| [M + H]⁺ | (wild- | ORF2 | ORF1 | compound | ing HPLC |
| (m/z) | type) | mutant | mutant | represented | peak |
| 386.1 | Absent | Present | Absent | | 1 |
| 260.2 | Present | Absent | Present | 2-heptyl-3- | 2 |
| | | | | hydroxy-4- | |
| | | | | quinolone (PQS) | |
| 286.2 | Present | Absent | Present | | 3 |
| 288.2 | Present | Absent | Present | | 4 |
| 272.2 | Present | Absent | Present | | 5 |
| 298.2 | Present | Absent | Present | N-(3- | 6 |
| | | | | oxododecanoyl)- | |
| | | | | L-homoserine | |
| | | | | lactone | |
| | | | | (C12-HSL) | |

Table 3 shows the principal ion masses of the peaks that differ between the wild-type and the ORF2* mutant P. aeruginosa PA14 strains. The HPLC peaks shown in FIGS. 12, 13, 15, and 17 that correspond to these compounds are indicated.

Further Analysis of the Signal that Inhibits Virulence

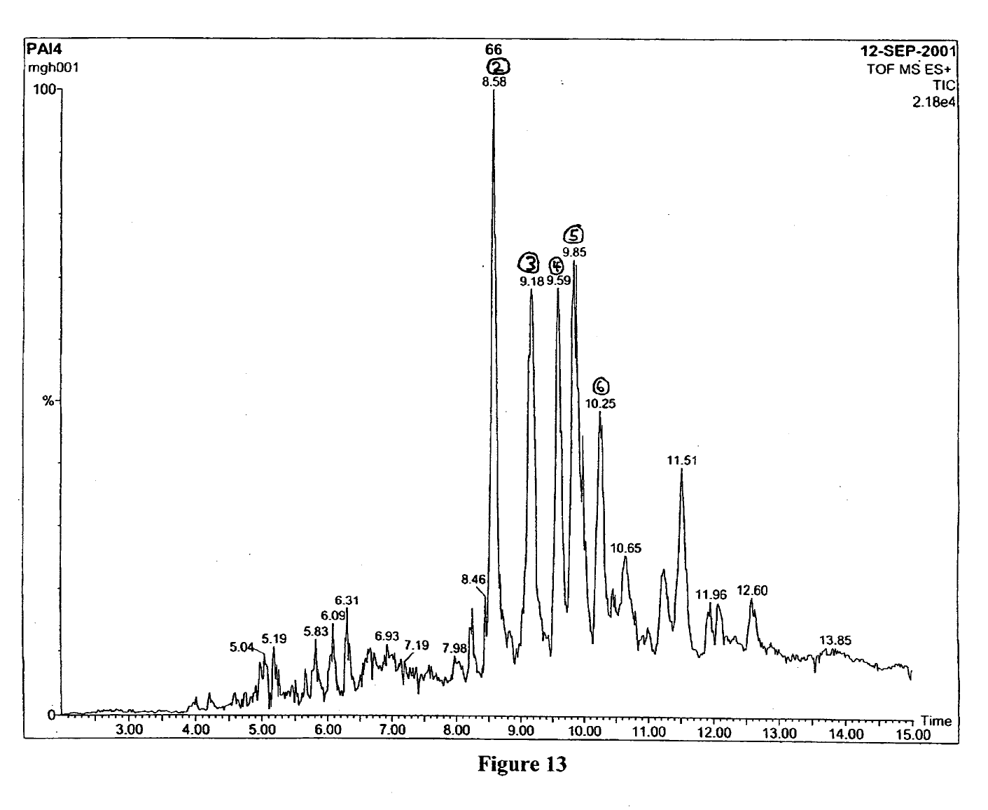

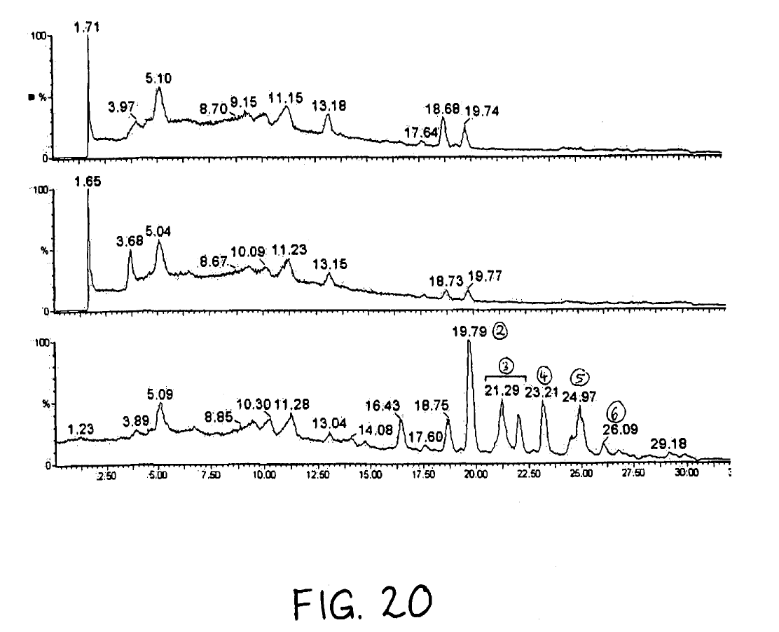

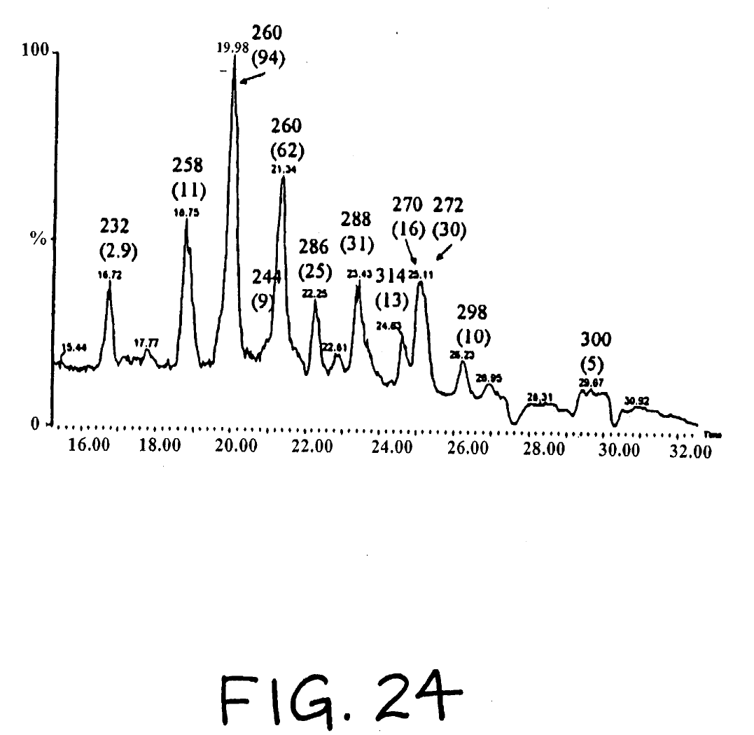

Inhibition of virulence, for example by the modulation of MvfR cleavage, is likely to be regulated by a component(s) present in the non-polar fraction of PA14 cells grown at the stationary phase that is not produced by the MvfR mutant. The supernatants from stationary phase LB cultures (OD₆₀₀=4.5-5.0) were extracted twice with ethyl acetate and the solvent concentrated under a stream of nitrogen gas. We then analyzed the organic extracts by liquid chromatography/mass spectrometry (LC/MS) using a reverse phase C₁₈ column coupled to a Quattro II triple quadrupole mass spectrometer in positive electrospray ionization mode. The chromatogram obtained from PA14 supernatant (FIG. 20, bottom) presented a large number of peaks (FIG. 24). The organic extracts of supernatants of mvfR and of the phnAB mutants grown at the stationary phase were also analyzed by LC/MS. Comparison of the chromatogram of mvfR (FIG. 20, middle) and phnAB mutants (FIG. 20, top) with the one from PA14 indicated that the wild type strain contains many peaks in the 15 to 30 minute range that are not present in the other two extracts. The compounds that contained in these peaks are likely to be the same as those contained in the corresponding peaks in FIG. 13.

These peaks were further analyzed in MS/MS mode and most of them were found to belong to at least five families of compounds as shown in Table 4. The congeners, within each series, present almost identical MS/MS spectra, which indicates that they only differ by the length of their aliphatic chain, which is cleaved preferentially in MS/MS.

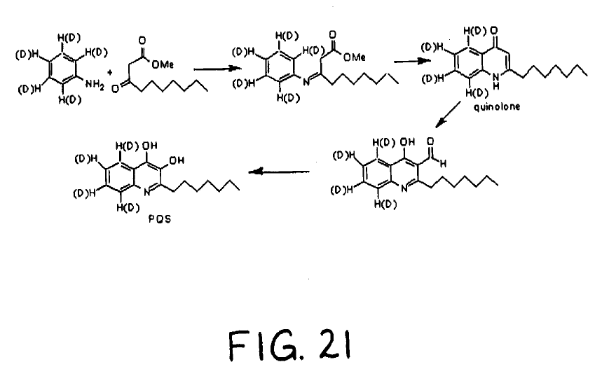

The compounds corresponding to the 244 and 272 ions of series 1 have been previously detected in P. aeruginosa cultures (Wells, J. Biol. Chem. 196:331-340, 1951). The structure of the compound corresponding to the 244 ions was elucidated by MS/MS and confirmed with the authentic compound synthesized according to standard methods. The 4-hydroxyquinoline standards were synthesized according to methods standard in the art. These methods included the synthesis of 2-heptyl-4-hydroxyquinoline by condensation of aniline and methyl 3-oxodecanoate, followed by cyclization. This intermediate was then transformed into a 3-formyl and into a 3-hydroxy compound (PQS). The same reactions were repeated with deuterated aniline to produce the corresponding deuterium-labeled compounds. Series 2 includes the Pseudomonas quinolone Signal (PQS) described by Pesci et al. (Proc. Natl. Acad. Sci. USA 96:11229-11234, 1999) in P. aeruginosa and the compound corresponding to the 260 ion was determined to be the actual PQS molecule by analysis of its MS/MS spectrum. This result was confirmed using synthetic PQS. (The synthesis method used is shown in FIG. 21.) Series 3 presents the same ions as those of series 1, but they appear at different retention times. MS/MS analyses of these ions show that they are N-oxides derivatives of the series 1. The compound corresponding to the 260 ions is commercially available and presents the same retention time and mass spectrum as the one observed in the extract. The series 4 and 5 are similar to the series 1 and 3, respectively, with the exception that the aliphatic chain contains an unsaturated bond.

TABLE 4
Structure of the compounds eluting between 15 and 30 minutes
in FIG. 20 and present in supernatants of PA14 cultures only.

| | | M + H | | |
|---|---|---|---|---|
| | | Ions | R = | |
| 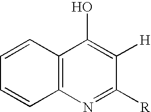 | Series 1 | 216 244 272 300 | C₅H₁₁ C₇H₁₅ C₉H₁₉ C₁₁H₂₃ | “PQS precursor” |
| 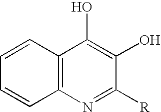 | Series 2 | 232 260 288 | C₅H₁₁ C₇H₁₅ C₉H₁₉ | PQS |
| 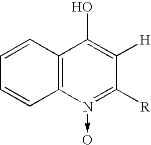 | Series 3 | 232 260 288 316 | C₅H₁₁ C₇H₁₅ C₉H₁₉ C₁₁H₂₃ | “N-oxide” |
| 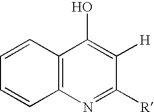 | Series 4 | 270 298 | C₉H₁₇ C₁₁H₂₁ | |
| 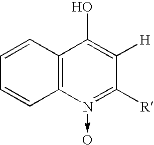 | Series 5 | 258 286 314 | C₇H₁₃ C₉H₁₇ C₁₁H₂₁ | |

From these experiments, we can conclude that a number of non-polar compounds are present in the supernatant of PA14 and absent in the supernatants of mvfR and phnAB mutants. Specifically, these results also show that inactivation of mvfR or phnAB completely inhibits the synthesis of PQS and of a wide variety of PQS-related compounds which are present in the organic extract of PA14 supernatant. In addition, the MS analysis confirmed that the autoinducers 3-oxo-C₁₂-HSL and C₄-HSL, and other unclassified compounds produced in very low abundance by PA14, are not present in the supernatant when mvfR is inactivated. This is in agreement with our results indicating that mvfR is involved in controlling the production of PQS, which likely occurs via regulation of the phnAB operon.

### The Effect of an Anti-Infective Compound on PA14 Virulence

Furthermore, given that the cleavage and release of MvfR protein can be induced by late stationary phase cell-free PA14-Sup, and that a knock-out of the mvfR gene has been shown to cause a reduction in virulence in both plant and animal hosts (Rahme et al., Proc. Natl. Acad. Sci. USA 94:13245-13250, 1997), we set out to determine if the premature cleavage and release of MvfR protein induced by PA14-Sup and PA14-AI affects the virulence of PA14. To address this issue, we added PA14-Sup and PA14-AI separately to PA14 cells grown to late exponential phase and the cells were allowed to grow for an hour. The cells were then centrifuged, re-suspended in 10 mM MgSO₄, and used for the evaluation of the morbidity and mortality in the thermal burn mouse model. PA14 strain grown in regular LB broth without any treatment was used as the control. The mortality caused by untreated PA14 in the thermal burn mouse model was 87.5% (as expected for wild type PA14) whereas the mortalities caused by PA14 cells pretreated with PA14-Sup and by bacteria pretreated with PA14-AI were 40% and 0%, respectively. Accordingly, the pretreatment of PA14 with PA14-Sup or PA14-AI reduces the virulence of the P. aeruginosa stain PA14. These results indicate that we identified a novel means to control P. aeruginosa infections.

In additional experiments, we added PA14-Sup and an ethyl acetate extract of the supernatant (PA14-OE, organic extract) (dissolved in LB after solvent removal) separately to PA14 cells grown to late exponential phase (OD₆₀₀=2.5-3.0) and the cells were incubated for one hour at 37° C. The cells were then centrifuged, re-suspended in 10 mM MgSO₄, and used for the evaluation of the morbidity and mortality in the burn mouse model. PA14 stain grown in regular Luria-Bertani (LB) broth without any treatment was used as the control. This experiment was repeated twice. The mortality caused by untreated PA14 was 75%±17 (std. dev.), as expected for wild type PA14, whereas the mortalities caused by PA14 cells treated with PA14-Sup and by bacteria pretreated with PA14-OE were 50%±9 (std. dev.), and 16%±16 (std. dev.), respectively. Similar to the untreated PA14 cells, PA14 cells pretreated with mvfR-Sup caused almost 85% mortality, indicating that mvfR is responsible for the production of the anti-infective compound(s). The above results demonstrate that compound(s) contained in the late stationary phase cell supernatants of PA14 and extractable by ethyl acetate, that may be responsible for the cleavage and release of the MvfR into the bacterial cell supernatant, may act as anti-infective compounds and, thereby, significantly attenuating the virulence of strain PA14.

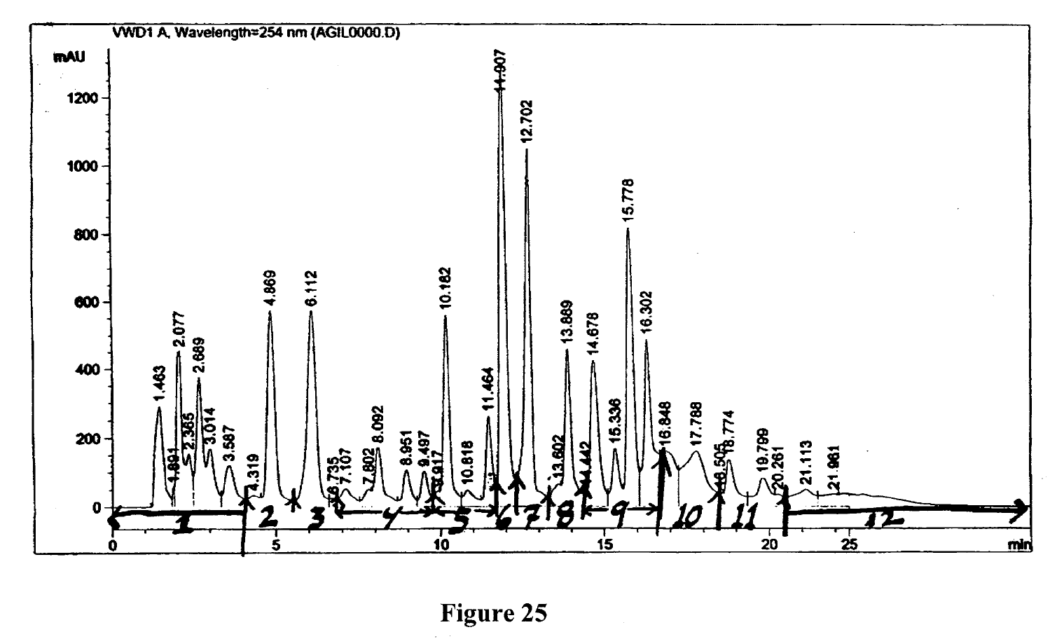

To isolate the non-polar compound present in PA14-AI that is acting as an anti-infective, the supernatant of PA14 cells grown in LB medium to late stationary phase (OD₆₀₀=4.5-5.0) was extracted twice with ethyl acetate and the solvent concentrated under a stream of nitrogen gas. The resulting organic extracts was fractionated by semi-preparative HPLC using a 150×4.5 mm C₁₈ column using UV detection. A water/acetonitrile gradient (with a constant concentration of 1% acetic acid) was used. The gradient started at 30% acetonitrile and terminated at 100% acetonitrile. Twelve fractions were recovered (FIG. 25). The first fraction contained the more polar compounds with a retention time of less than 15 minutes, the next ten fractions included all the peaks observed between 15 and 30 minutes, and a final fraction contained the non-polar compounds with retention times longer than 30 minutes. The solvent was removed and, to reduce the number of animals required to test each of these fractions in the burn mouse model separately, some fractions were pooled together. The P. aeruginosa strain PA14 cells used for infection were grown to the late exponential phase (OD₆₀₀=3.0), washed and exposed to the solvent-extracted fractions in combinations: fractions 1 to 4, fractions 5 to 8, and fractions 9 to 12, as well as a pool of the 12 fractions. The cells were then incubated at 37° C. for 1 hour before infection. The pool composed of the fractions 5 to 8 was found to contain significant anti-infective activity and, accordingly, contains an anti-infective compound. The compounds in this pool include the following compounds listed in Table 4: the N-oxide (peak at 10.182 minutes in FIG. 25), the PQS precursor (peak at 11.464 minutes in FIG. 25), PQS (peak at 11.907 minutes in FIG. 25), an N-oxide analog where R=C₉H₂₃ (peak at 12.702 minutes in FIG. 25), and a compound where R=C₁₁H₂₁ (peak at 13.889 minutes in FIG. 25). One of these compounds, alone or in combination with another, is likely to be an anti-infective compound. In addition, a compound found in one of the other fractions may further enhance the anti-infective activity of a compound present in the pool of fractions 5 to 8.

### Additional Functions of MvfR

To obtain additional information about the function of MvfR from its sequence, we performed a PSI-BLAST search. This search revealed that the MvfR protein contains an atrial natriuretic peptide (ANF) binding domain spanning the region between amino acids 87-293 of the sequence shown in FIG. 10. The ANF domain is a ligand-binding domain present in a wide range of eukaryotic receptors, as well as in bacterial amino acid binding proteins responsible for the transport of branched-chain amino acids. For example, the Escherichia coli LIV-I and LS amino acid transport systems are high-affinity, periplasmic, binding protein-dependent systems that utilize the leucine-, isoleucine-, valine-binding protein (LIV-BP) and leucine-specific binding protein (LS-BP), respectively. These two binding proteins (BPs), when first translated, contain a 23-amino acid signal sequence that is removed when the BPs are secreted into the periplasm where they carry out their function. When the receptor protein binds a substrate, a conformational change occurs within the protein, which allows for the recognition of the substrate-bound receptor by the transport system and for the subsequent deposit of the substrate into the cytoplasm of the bacteria. Moreover, the ligand-induced conformational change mechanism of these receptors is also implicated in the process of chemotaxis. In light of the similarities that the MvfR protein architecture shares with these E. coli proteins, it is likely that MvfR also is involved in binding and/or translocation of small molecules including sugars, amino acids, and peptides.

The above experiments were carried out using the following materials and methods.

### Materials and Methods

### Bacterial Strains, Media and Growth Conditions

The strains used for this study were the following: E. coli strains, TOP 10, BL21 (DE3); Pseudomonas aeruginosa strains: UCBPP-PA14 (deposited with the American Type Culture Collection (ATCC) on Mar. 22, 1995, and bears accession number 55664), TnphoA-mutagenized UCBPP-PA14 mutant strain (Rahme et al., Science 268:1899-1902, 1995), pho34B12 (Rahme et al., Proc. Natl. Acad. Sci. USA 94:13245-13250, 1997), PAO1 (Holloway, J. Gen. Microbiol. 13:572-581, 1955), and PAO1 isogenic mutant strains, lasR⁻, rhlR⁻ and lasR⁻rhlR⁻ (Pearson, et al., J. Bacteriol. 179:5756-5767, 1997). The E. coli and P. aeruginosa strains were grown at 37° C. in Luria-Bertani (LB) medium.

With regard to the ATCC deposit of P. aeruginosa strain UCBPP-PA14, applicants acknowledge their responsibility to replace this strain should it loose viability before the end of the term of a patent issued hereon, and their responsibility to notify the ATCC of the issuance of such a patent, at which time the deposit will be made available to the public. Prior to that time the deposit will be made available to the Commissioner of Patents under the terms of 37 C.F.R. §1.14 and 35 U.S.C. §112.

### Site-Directed Mutagenesis

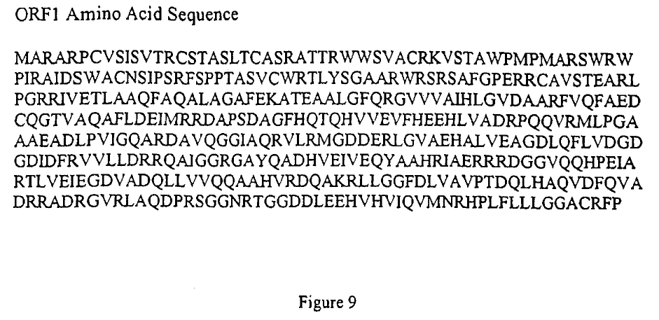

The plasmid pLGR34B12 (Rahme et al., Proc. Natl. Acad. Sci. USA 94:13245-13250, 1997), with a 3.7-kb EcoRI fragment containing the entire pho34B12 locus, was used for mutagenesis. In the pho34B12 region, ORF1 and ORF2 are arranged in such a way that the first nucleotide of an ORF1 codon is the third nucleotide of an ORF2 codon and vise versa. Accordingly, the point mutations were introduced via polymerase chain reaction (PCR) into the first nucleotide of a codon in both ORFs. In ORF1, a codon for glutamine (amino acid 262 of the sequence shown in FIG. 9) was converted to a stop codon by changing CAG to TAG and in ORF2, a codon for glutamic acid (amino acid 151 of the sequence shown in FIG. 10) was switched to a stop codon by changing GAG to TAG. The single nucleotide change in each ORF was confirmed by sequencing. The mutagenized 3.7-kb EcoRI fragments were then sub-cloned into the SmaI site of pCVD (Donnenberg and Kaper, Infect. and Immunity 59:4310-4317, 1991) to generate two plasmids containing mutated ORF1 (pHCORF1*) and mutated MvfR (pHCORF2*). These two plasmids were then used to replace the pho34B12 gene with the TnphoA insertion via homologous recombination as described in Donnenberg and Kaper (Infect. and Immunity 59:4310-4317, 1991). The resulting mutants are designated as ORF1* and ORF2*, respectively.

### Virulence Studies in Plant and Animal Models

The assessment of P. aeruginosa growth in Arabidopsis and the determination of mortalities in the thermal injury mouse model were performed as previously described in Rahme et al. (Proc. Nail. Acad. Sci. USA 94:13245-13250, 1997).

### Analysis of Pyocyanin Level

Quantification of the level of pyocyanin in cell-free P. aeruginosa cultures was performed as described in Essar et al. (J. Bacteriol. 172:884-900, 1990).

Construction of mvfR-lacZ and phnAB-lacZ Transcriptional Fusions

To construct the mvfR-lacZ fusion, a 473-bp fragment of mvfR (447-bp upstream and 26-bp downstream of the start codon) was PCR amplified and cloned into the EcoRI/BamHI site of pPCS1002 (Albus et al., J. Bacteriol. 179:3928-3935, 1997) to obtain pHCmvfR-lacZ. To construct the phnAB-lacZ fusion, a 522-bp fragment phnAB (478-bp upstream and 44-bp downstream of the start codon of phnA) was PCR amplified and cloned into the EcoRI/BamHI site of pPCS1002 to obtain pHCphnAB-lacZ. The plasmid pHCmvfR-lacZ was transformed into P. aeruginosa strains PA14 and ORF2*. In addition, the plasmid pHCphnAB-lacZ was introduced into strains PA14 and ORF2*.

### β-Galactosidase Assays

P. aeruginosa strains containing the lacZ transcriptional fusions were grown overnight to OD₆₀₀=2.5-3.0 or as indicated. The β-galactosidase assays were carried out as described in Miller (“Experiments in Molecular Genetics,” Cold Spring Harbor Laboratory Press, Cold Spring Harbor, N.Y. (1972)).

Overexpression and Purification of MvfR in E. coli

A 1035-bp fragment containing the entire coding region of mvfR was PCR amplified and cloned into the XhoI/HindIII site of pBAD/HisA (Invitrogen, Carlsbad, Calif.) to obtain pBAD/His-mvfR. To overexpress MvfR, an overnight culture of an E. coli strain containing pBAD/His-mvfR was diluted 1:10 into fresh LB medium and grown to an OD₆₀₀=0.5. Arabinose was then added to the culture at a final concentration of 0.02% and the culture was grown at 37° C. for another 4 hours. The purification of recombinant MvfR was performed according to the instructions provided with the XPRESS™ System protein purification kit (Invitrogen, Carlsbad, Calif.). The purified recombinant protein was then treated with enterokinase to remove the N-terminal part of the amino acids that do not belong to MvfR. Once purified, the MvfR protein was used in a gel electrophoresis mobility shift assay.

### Gel Electrophoresis Mobility Shift Assay

We synthesized two pairs of complementary oligonucleotides from the promoter region of the phnAB operon. One pair is 210-160 by (P1) upstream of the methionine start codon, and the other is 484-434 by (P2) upstream. About 100 ng of each oligonucleotide pair were annealed and radio-labeled DNA was obtained by end-labeling with ³²P using T4 polynucleotide kinase and [γ-³²P]ATP. Approximately 4000 cpm of the radio-labeled probes and 0.5 ng of the purified mvfR protein were used for each DNA-binding reaction. The DNA-binding assays were carried out at room temperature for 25 min in 20 mM Tris-HCl (pH 7.5), 50 mM KCl, 2 mM MgCl₂, 1 mM DTT, 5% glycerol, 10 ng/μl poly(dI.dC), 10 ng/μl bovine serum albumin and 1:500 dilution of the protease inhibitor cocktail III from Calbiotech. The reaction mix was immediately loaded onto a 6% polyacrylamide gel and electrophoresis was performed in 0.5×TBE buffer at 135 V for 4 hours. The gel was then dried and exposed on X-ray film.

Construction of the mvfR-GST Translational Fusion

An approximately 1.5 kb fragment containing both the promoter and the entire coding region (excluding the stop codon) of mvfR, an approximately 0.7 kb fragment containing the entire coding region of GST gene from Schistosoma japonicum, and an approximately 0.6 kb fragment of the mvfR 3′ region downstream of the stop codon were cloned into HindIII/XbaI, XbaI/KpnI, and KpnI/EcoRI sites of the pUCP19 vector (West et al., Gene 148:81-86, 1994), respectively, to obtain pUCP19/mvfR-GST. The pUCP19/mvfR-GST was then introduced into P. aeruginosa strains via electroporation as previously described in Bloemberg et al. (Microbiol. 63:4543-4551, 1997).

### Cell Fractionation

Bacterial strains were grown to the indicated optical density and pelleted. Cell fractionations were performed as described in Thomas et al. (J. Bacteriol. 174:6771-6779, 1992). The additional fractionation of the membrane into inner and outer membranes was carried out using sucrose gradient centrifugation as described in Hancock and Nikaido (J. Bacteriol. 136:381-390, 1978). The separation of outer membrane from inner membrane was monitored by measuring the activities of NADH oxidase (inner membrane marker) in each membrane fraction as described by Osborn et al. (J. Biol. Chem. 247:3962-3972, 1972).

### Immunoblot Analysis

Proteins of cell fractions were separated on 10% SDS-polyacrylamide gels and transferred to IMMOBILON-P membranes (Millipore, Bedford, Mass.) following the manufacturer's instructions. The immunoblot analyses were performed with an anti-GST monoclonal antibody following manufacturer's instructions (Upstate Biotechnology, Lake Placid, N.Y.).

Extraction and Liquid Chromatography/Mass Spectrometry Analysis of P. aeruginosa Auto Inducers

Cultures of wild-type P. aeruginosa strain PA14, or P. aeruginosa strain PA14 mutant for ORF2 or ORF1 were started from a frozen stock of bacteria using 7 mls media with no antibiotics. Cultures were grown overnight at 37° C. with shaking. Once the A₆₀₀ was at least 3.5, 5 mls of the cultures were spun down at 4000×g for 10 minutes. The supernatant was transferred to a 10 ml syringe and, using a 0.2 μm filter, filtered into clean glass test tube (without plastic cap). Starting with this step of the protocol, we eliminated the use of plasticware and used only clean glass to extract the compounds. The supernatant was extracted twice with 5 mls ethyl acetate (HPLC grade) in a clean and completely dry 30 ml separatory funnel. The ethyl acetate fractions were pooled in a 25 ml flask, discarding the aqueous phase as waste. Approximately 0.5 g anhydrous MgSO₄ was added to the ethyl acetate fractions to remove any remaining moisture in the samples. The ethyl acetate fractions were decanted through a funnel lined with Whatman 1 filter paper and the filtrate was collected in a 10 ml beaker. The individual decanted fractions in the beaker were evaporated to dryness under a stream of N₂ gas. The dry fractions were reconstituted in 200 μl ethyl acetate using a Hamilton syringe, and transferred to a silanized glass vial with sterile Al-lined cap.

Following the extraction of the autoinducers, the samples were dried further and either stored at −20° C., or re-dissolved in 20 μl 50% acetonitrile/water and subjected to Liquid Chromatography (LC)/Mass Spectrometry (MS) analysis. These analyses were performed with a LCT mass spectrometer (Micromass Inc., Beverly, Mass.) using electrospray in the positive mode, interfaced with an HP 1100 liquid chromatograph. Samples were eluted using a gradient of water/0.1% formic acid to acetonitrile/0.1% formic acid, over a 15 min. period. The columns used were GENESIS reversed-phase C₁₈ columns (150 mm, 2.1 mm internal diameter, 4 μm particle size). The scanning mass range spanned from 200 to 1450 daltons. Under the operating parameters used, little fragmentation of the pseudomolecular ions was expected.

### Example 2

### Analysis of Compounds from P. aeruginosa Cultures which May be Used to Treat or Prevent Bacterial or Fungal Infections

We have previously demonstrated that the MvfR protein controls the production of pyocyanin, elastase, phospholipase, autoinducer I (3-oxo-dodecanoyl homoserine lactone) and the PQS as well as the levels of various P. aeruginosa secreted proteins, strongly indicating that MvfR is a QS-related regulator. Moreover, expression studies with QS regulators lasR, rhlR, and gacA indicate that, at the transcriptional level, mvfR operates independently of these regulators and that these regulators operate independently of mvfR, even though they all play a role in the regulation of pyocyanin production and other QS-dependent components. However, the results of the expression studies do not exclude the possibility that mvfR may interact at the post-transcriptional level with these QS regulators.

We have also previously demonstrated that the MvfR protein regulates the expression of phnAB operon. This latter operon encodes an anthranilate synthase (Essar et al., J. Bacteriol. 172:884-900, 1990). Since anthranilic acid has been considered for a long time a precursor for pyocyanin production, and activation of phnAB resulted in a reduced production of pyocyanin (Essar et al., J. Bacteriol. 172:884-900, 1990; Mahajan-Miklos et al., Cell 96:47-56, 1999), it is generally considered that phnAB is part of the synthetic pathway of pyocyanin (Essar et al., J. Bacteriol. 172:884-900, 1990). However, it was recently demonstrated that anthranilic acid is, in fact, not a precursor of the phenazine nucleus (Mavrodi et al., J. Bacteriol. 183:6454-6465, 2001; McDonald et al., J. Am. Chem. Soc. 123:9459-9460, 2001). Interestingly, the study of the biosynthetic pathway of the PQS has revealed that anthranilic acid is a precursor of this important signaling molecule produced by P. aeruginosa (Calfee et al., Proc. Natl. Acad. Sci. USA 98:11633-11637, 2001). The phnAB operon is also involved in the production of pyocyanin.

We have developed an efficient method to quantitate PQS and PQS-related compounds to study the biological activity of the PA14 supernatant extract. Prior to our developing this method, there was no such simple analytical technique, other than indirect biological assays (McKnight et al., J. Bacterial. 182:2702-2708, 2000). The similarities between the various compounds observed in PA14 supernatant suggest a common origin. More specifically, the series 1 could well be the precursor of the series 2 and 3 by simple oxidation at the 3 position or on the nitrogen of the compounds of series 1 (Table 4).

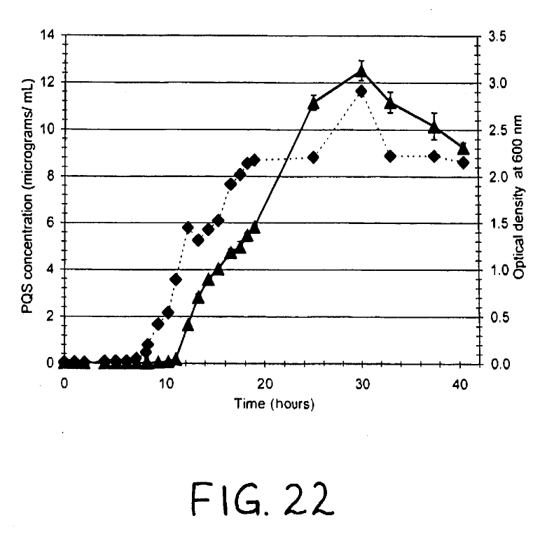

We used deuterium-labeled PQS to make a mass spectrometric stable isotope dilution assay to quantitate PQS in growing cultures of P. aeruginosa. PQS concentrations are presented along with the optical densities (OD) of the cultures (FIG. 22). These analyses show that PQS is produced late during the growth phase.

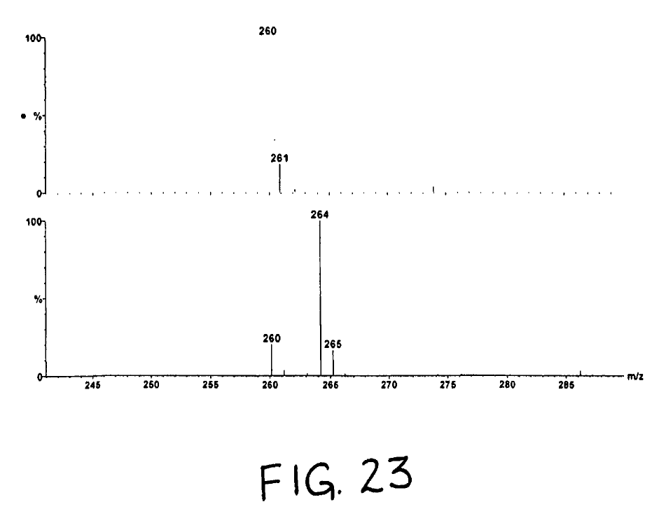

While, using radiolabeled compounds, anthranilic acid has been shown to be a precursor of PQS (Chugani et al., Proc. Natl. Acad. Sci. USA 98:2752-2757, 2001), there have been no reported studies showing other intermediates in the biosynthesis of PQS. To begin characterizing our proposed biosynthetic pathway, we established that the compound corresponding to the 244 ion of the series 1 (Table 4) is a precursor of PQS. The labelled putative PQS precursor shown in FIG. 21 was added to the growth medium at a concentration of 5 μg/ml before starting the cultures. After 15 hours of incubation, the MS spectrum of PQS showed more than 80% label incorporation (FIG. 23). These results demonstrate that the putative PQS precursor is indeed part of the biosynthetic pathway of PQS. LC/MS analyses of P. aeruginosa cultures show that the cultures contain a large number of 4-hydroxyquinolines, including 2-alkyl-4-hydroxyquinoline and 2-alkyl-4-hydroxyquinoline-N-oxide. This finding provides important information about the enzymes involved in the pathway and eventually can lead to the synthesis of inhibitors of PQS biosynthesis.

Based on the biosynthesis pathway shown herein and the enzymes that are presumed to be part of this pathway, the five genes directly upstream of phnAB are likely to be involved in the synthesis of PQS (and related compounds): PA0996, PA0997, PA0998, PA0999, and PA1000 (numbers correspond to the PAO1 annotation). Data from the genechip analysis (functional genomics) show that these genes are co-regulated by MvfR. For example, the PA0996 protein appears to be involved in the coupling of the anthranilic acid and the PA0999 could present the 3-keto esters to the anthranilate moiety.

The above experiments were carried out using the following materials and methods.

### Materials and Methods

To quantify PQS in growing cultures of P. aeruginosa, we used deuterium-labeled PQS for a mass spectrometric stable isotope dilution assay. The mass spectrometer was a Micromass Quattro II interfaced to an Agilent HP1100 sample handling system equipped with a 150×4.6 mm C18 reverse phase column. A 35 minute water/acetonitrile gradient (with 1% acetic acid) was used. Analyses were performed in positive electrospray. Strain PA14 was cultivated in Luria broth at 37° C. Labeled and unlabeled PQS and PQS precursor were synthesized as shown in FIG. 21 using deuterium-labeled aniline as the starting material. Quantification of PQS was performed using labelled PQS in a stable isotope dilution assay. The cultures were sampled at regular intervals, known amounts of labelled PQS were added, and the samples were centrifuged and injected. The analyses were performed in Selective Ion Recording (SIR) mode by monitoring the m/z 260 (PQS) and 264 (labelled PQS) ions to obtain a direct quantification of PQS in the culture. A calibration curve was performed for PQS. The calibration curve was linear between 0.1 and 40 μg/ml. Quantification was performed over a period of 41 hours.

### Example 3

### Test Extracts and Compounds

In general, compounds that affect MvfR cleavage or expression may be identified from large libraries of both natural products, synthetic (or semi-synthetic) extracts or chemical libraries, according to methods known in the art.

Those skilled in the art will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modifications of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from, for example, Brandon Associates (Merrimack, N.H.) and Aldrich Chemical (Milwaukee, Wis.).

Alternatively, libraries of natural compounds in the form of bacterial, fungal, plant, and animal extracts are commercially available from a number of sources, including, but not limited to, Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art (e.g., by combinatorial chemistry methods or standard extraction and fractionation methods). Furthermore, any isoform of a compound, i.e., the D or L isoform, may be used, and, if desired, any library or compound may be readily modified using standard chemical, physical, or biochemical methods.

In addition, those skilled in the art readily understand that methods for dereplication (e.g., taxonomic dereplication, biological dereplication, and chemical dereplication, or any combination thereof) or the elimination of replicates or repeats of materials already known for their effects on compounds associated with bacterial infections should be employed whenever possible.

When a crude extract is found to affect MvfR cleavage or expression, further fractionation of the positive lead extract may be carried out to further isolate chemical constituents responsible for the observed effect. For example, as is noted above, using mass spectrometry, we identified five compounds that are absent in the ORF2* (mvfR) mutant P. aeruginosa strain PA14, but are present in the wild-type P. aeruginosa strain PA14, as well as one that is present in the ORF2* (mvfR) mutant but is absent in the wild-type. One skilled in the art would know how to further characterize these compounds to identify which of these chemical entities affect MvfR cleavage. The same in vivo and in vitro assays described herein for the detection of activities in mixtures of compounds can be used to purify the active component and to test derivatives thereof. Methods of fractionation and purification of such heterogenous extracts are known in the art.

Assays to be used for identifying compounds that affect MvfR cleavage may include measuring the amount of cleaved MvfR protein using polyacrylamide gel electrophoresis or Western analysis.

### Example 4

Prevention and Treatment of Bacterial and Fungal Infections

Compounds identified as affecting MvfR cleavage or expression, for example, the L and/or D isoforms of C₁₂-HSL, C₄-HSL, or PQS, may be used in treating a bacterial infection, for example, a P. aeruginosa infection, or a fungal infection, for example, a yeast infection, e.g., a Candida albicans or Saccharomyces cerevisiae infection, or a Cryptococcus neoformans or Fusarium oxysporum infection, in an animal. In addition, an anti-fungal compound may be used to treat an infection of a plant by a fungal pathogen (e.g., Fusarium oxysporum). Such therapeutic compounds may be administered by any of a variety of routes known to those skilled in the art, e.g., by intraperitoneal, subcutaneous, parenteral, intravenous, intramuscular, or subdermal injection. However, a therapeutic compound may also be administered as an aerosol, as well as orally, nasally, or topically. Appropriate carriers or diluents, as well as what is essential for the preparation of a pharmaceutical composition, are described, e.g., in Remington's Pharmaceutical Sciences (18^{th} edition), ed. A. Gennaro, 1990, Mack Publishing Company, Easton, Pa., a standard reference book in this field.

Formulations for parenteral administration may, for example, contain excipients, sterile water, or saline. In addition, for inhalation, an excipient may be, for example, lactose. Furthermore, aqueous solutions may be used for administration in the form of nasal drops, or as a gel for topical administration. The exact dosage used will depend on the severity of the condition (e.g., the level of bacterial infection), or the general health of the patient and the route of administration. The therapeutic compound may be administered once, or it may repeatedly be administered as part of a regular treatment regimen over a period of time.

In addition, the test compounds, for example, the L and/or D isoforms of C₁₂-HSL, C₄-HSL, or PQS, may also be used to treat a bacterial infection, for example a Pseudomonas infection, or a fungal infection in any of a number of plant cells or whole plant including, without limitation, algae, tree species, ornamental species, temperate fruit species, tropical fruit species, vegetable species, legume species, crucifer species, monocots, dicots, or in any plant of commercial or agricultural significance. Examples of such plants include, but are not limited to, conifers, petunia, tomato, potato, pepper, tobacco, Arabidopsis, grape, lettuce, sunflower, oilseed rape, flax, cotton, sugarbeet, celery, soybean, alfalfa, Medicago, lotus, Vigna, cucumber, carrot, eggplant, cauliflower, horseradish, morning glory, poplar, walnut, apple, grape, asparagus, cassava, rice, maize, millet, onion, barley, orchard grass, oat, rye, and wheat.

For the treatment or prevention of a bacterial or a fungal infection in a plant, a compound capable of cleaving MvfR, or affecting the expression of MvfR, may be topically administered, transiently or stably expressed, or produced in a transgenic plant. A number of vectors suitable for stable or extrachromosomal transfection of plant cells or for the establishment of transgenic plants are available to the public; such vectors are described in Pouwels et al. (Cloning Vectors: A Laboratory Manual, 1985, Supp. 1987), Weissbach and Weissbach, (Methods for Plant Molecular Biology, Academic Press, 1989), and Gelvin et al. (Plant Molecular Biology Manual, Kluwer Academic Publishers, 1990).

Typically, plant expression vectors include a cloned plant gene under the transcriptional control of 5′ and 3′ regulatory sequences and a dominant selectable marker. Such plant expression vectors may also contain, if desired, a promoter regulatory region (for example, one conferring inducible or constitutive, pathogen- or wound-induced, environmentally- or developmentally-regulated, or cell- or tissue-specific expression), a transcription initiation start site, a ribosome binding site, an RNA processing signal, a transcription termination site, and/or a polyadenylation signal.

### Other Embodiments

From the foregoing description, it will be apparent that variations and modifications may be made to the invention described herein to adapt it to various usages and conditions. Such embodiments are also within the scope of the following claims.

All publications mentioned in this specification are herein incorporated by reference to the same extent as if each independent publication or patent application was specifically and individually indicated to be incorporated by reference.

What is claimed is:

1. A method of treating or stabilizing a P. aeruginosa infection in a mammal, said method comprising administering to said mammal a compound that promotes the modification of an MvfR protein in an amount sufficient to treat or stabilize said infection, wherein said compound is selected from the group consisting of (a) N-butyryl-L-homoserine lactone, 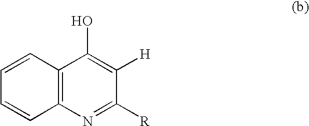
where R=C₅H₁₁, C₇H₁₅, C₉H₁₉, or C₁₁H₂₃ and the compound has an M+H ion of 216, 244, 272, or 300 daltons; 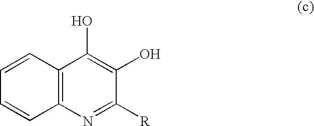
where R=C₅H₁₁, C₇H₁₅, or C₉H₁₉ and the compound has an M+H ion of 232, 260, or 288 daltons; 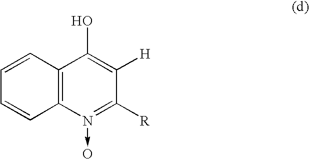
where R=C₅H₁₁, C₇H₁₅, C₉H₁₉, or C₁₁H₂₃ and the compound has an M+H ion of 232, 260, 288, or 316 daltons; 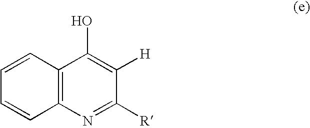
where R′=C₉H₁₇ or C₁₁H₂₁ and the compound has an M+H ion of 270 or 298 daltons; and 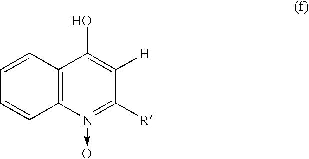
where R′=C₇H₁₃, C₉H₁₇, or C₁₁H₂₁ and the compound has an M+H ion of 258, 286, or 314 daltons.

2. The method of claim 1, wherein said compound is N-butyryl-L-homoserine lactone.

3. The method of claim 1, wherein said compound is selected from the group consisting of 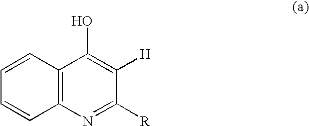
where R=C₅H₁₁, C₇H₁₅, C₉H₁₉, or C₁₁H₂₃ and the compound has an M+H ion of 216, 244, 272, or 300 daltons; 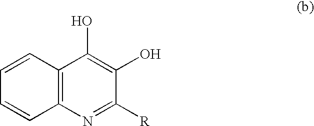
where R=C₅H₁₁, C₇H₁₅, or C₉H₁₉ and the compound has an M+H ion of 232, 260, or 288 daltons; 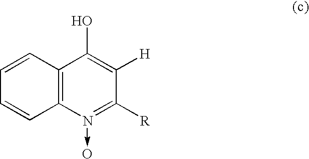
where R=C₅H₁₁, C₇H₁₅, C₉H₁₉, or C₁₁H₂₃ and the compound has an M+H ion of 232, 260, 288, or 316 daltons; 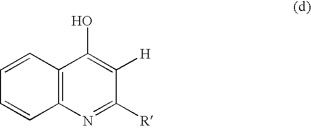
where R′=C₉H₁₇ or C₁₁H₂₁ and the compound has an M+H ion of 270 or 298 daltons; and 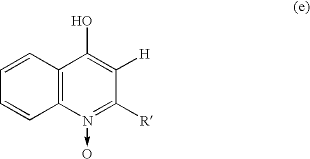
where R′=C₇H₁₃, C₉H₁₇, or C₁₁H₂₁ and the compound has an M+H ion of 258, 286, or 314 daltons.

4. The method of claim 1, wherein said mammal is immuno-compromised.

5. The method of claim 1, wherein said MvfR protein is cleaved.

6. The method of claim 5, wherein said MvfR protein is cleaved between amino acids 146 and 147 of the sequence of SEQ ID NO:2.

7. The method of claim 5, wherein said cleavage of said MvfR protein results in a polypeptide fragment with a molecular weight of approximately 22 kDa.

8. The method of claim 1, wherein said mammal is a human.

9. The method of claim 8, wherein said human has cystic fibrosis.

10. A method of treating or stabilizing a Candida albicans, Cryptococcus neoformans, or Fusarium oxysporum infection in a mammal, said method comprising administering to said mammal a compound that promotes the modification of an MvfR protein in an amount sufficient to treat or stabilize said infection, wherein said compound is selected from the group consisting of (a) N-(3-oxododecanoyl)-L-homoserine lactone, (b) N-butyryl-L-homoserine lactone, (c) 2-heptyl-3-hydroxy-4-quinolone, 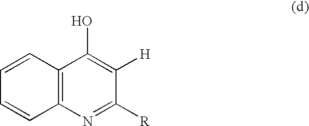
where R=C₅H₁₁, C₇H₁₅, C₉H₁₉, or C₁₁H₂₃ and the compound has an M+H ion of 216, 244, 272, or 300 daltons; 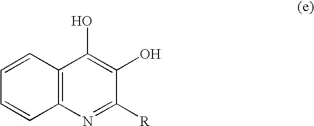
where R=C₅H₁₁, C₇H₁₅, or C₉H₁₉ and the compound has an M+H ion of 232, 260, or 288 daltons; 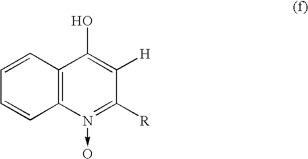
where R=C₅H₁₁, C₇H₁₅, C₉H₁₉, or C₁₁H₂₃ and the compound has an M+H ion of 232, 260, 288, or 316 daltons; 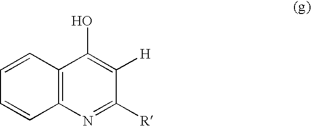
where R′=C₉H₁₇ or C₁₁H₂₁ and the compound has an M+H ion of 270 or 298 daltons; and 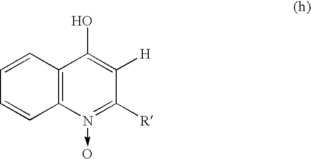
where R′=C₇H₁₃, C₉H₁₇, or C₁₁H₂₁ and the compound has an M+H ion of 258, 286, or 314 daltons.

11. A method of identifying a candidate compound for the treatment of a fungal or bacterial infection, said method comprising the steps of:
(a) contacting a cell expressing an MvfR protein with a candidate compound; and
(b) measuring the amount of modified MvfR protein; an increase in modified MvfR protein indicating that said candidate compound modified MvfR protein.


SEQUENCE LISTING (3 sequences)

SEQ ID NO: 1 - protein, 427 aa, Pseudomonas aeruginosa
MARARPCVSISVTRCSTASLTCASRATTRWWSVACRKVSTAWPMPMARSWRWPIRAIDSWACNSIPSRFSPPTASVCWRTLYSGAARWRSRSAFGPERRCAVSTEARLPGRRIVETLAAQFAQALAGAFEKATEAALGFQRGVVVAIHLGVDAARFVQFAEDCQGTVAQAFLDEIMRRDAPSDAGFHQTQHVVEVFHEEHLVADRPQQVRMLPGAAAEADLPVIGQARDAVQGGIAQRVLRMGDDERLGVAEHALVEAGDLQFLVDGDGDIDFRVVLLDRRQAIGGRGAYQADHVEIVEQYAAHRIAERRRDGGVQQHPEIARTLVEIEGDVADQLLVVQQAAHVRDQAKRLLGGFDLVAVPTDQLHAQVDFQVADRRADRGVRLAQDPRSGGNRTGGDDLEEHVHVIQVMNRHPLFLLLGGACRFP

SEQ ID NO: 2 - protein, 344 aa, Pseudomonas aeruginosa
MPIHNLNHVNMFLQVIASGSISSAARILRKSHTAVSSAVSNLEIDLCVELVRRDGYKVEPTEQALRLIPYMRSLLNYQQLIGDIAFNLNKGPRNLRVLLDTAIPPSFCDTVSSVLLDDFNMVSLIRTSPADSLATIKQDNAEIDIAITIDEELKISRFNQCVLGYTKAFVVAHPQHPLCNASLHSIASLANYRQISLGSRSGQHSNLLRPVSDKVLFVENFDDMLRLVEAGVGWGISPHYFVEERLRNGTLAVLSELYEPGGIDTKVYCYYNTALESERSFRRFLESARQRLRELGRQRFDDAPAWQPSLGRNCAAPLRPEGAPRPPTRRTRVERSPADAGRGW

SEQ ID NO: 3 - DNA, 1659 bp, Pseudomonas aeruginosa
tgcgcaacgatacgccgttgccgacgatccaggcggcattgctggccgacggttgcgaactgctggtgctgtcgccggggcccggtcggccggaagacgccggctgtatgctggaattgctcgcctgggcccgcgggcgcttgccggtgctcggcgtctgcctcggccaccaggcgctggcgctggccgccggtggcgcggtgggcgaggcgaggaagccgctgcatggcaagagcacgtccctgcgtttcgatcagcgtcacccgctgttcgacggcatcgctgacctgcgcgtcgcgcgctaccactcgctggtggtcagtcgcctgccggaaggtttcgactgcctggccgatgccgatggcgagatcatggcgatggccgatccgcgcaatcgacagctgggcttgcaattccatcccgagtcgattctcaccacccacggccagcgtctgctggagaacgctctactctggtgcggcgcgttggcggtcgcggagcgccttcgggcctgagcggcgctgcgcagtttcgaccgaggctcggttgccaggccggcgcatcgtcgaaacgctggcggcccagttcgcgcaggcgctggcgggcgctttcgagaaagcgacggaagctgcgctcggattccagcgcggtgttgtagtagcaatacaccttggtgtcgatgccgcccggttcgtacagttcgctgaggactgccagggtaccgttgcgcaggcgttcctcgacgaaataatgcggcgagatgccccatccgacgccggcttccaccagacgcagcatgtcgtcgaagttttccacgaagagcaccttgtcgctgaccggccgcagcaggttcgaatgctgcccggagcggctgccgaggctgatctgccggtaattggccaggctcgcgatgctgtgcagggaggcattgcacaacgggtgctgcggatgggcgacgacgaacgccttggtgtagccgagcacgcactggttgaagcgggagatcttcagttcctcgtcgatggtgatggcgatatcgatttccgcgttgtcctgcttgatcgtcgccaggctatcggcgggcgaggtgcgtatcaggctgaccatgttgaaatcgtcgagcagtacgctgctcaccgtatcgcagaacgacggcgggatggcggtgtccagcagcacccggagattgcgcggacccttgttgagattgaaggcgatgtcgccgatcagctgctggtagttcagcaggctgcgcatgtaagggatcaggcgaagcgcctgctcggtgggttcgaccttgtagccgtcccgacggaccagctccacgcacaggtcgatttccaggttgctgaccgccgagctgaccgcggtgtgcgacttgcgcaggatccgcgcagcggaggaaatcgaaccggaggcgatgacctggaggaacatgttcacgtgattcaggttatgaataggcatcccttattccttttattgggtggcgcgtgccgcttcccttgatcgggtcaggttgccgctactgtggaagaagcgtcgaggactcgatagatagcgcccgagtgtttcaacttgtcttctggatgacgttttcatcggggaaacctcccgtcggtcagtgaatcgcaagggctggcgtgcgagggtggaatc